(19) United States
(12) Patent Application Publication
(10) Pub. No.: US 2022/0308052 A1
(43) Pub. Date: Sep. 29, 2022

(54) COMPOSITIONS AND METHODS FOR DETECTING AUTOANTIBODIES

(71) Applicants: THE JOHNS HOPKINS UNIVERSITY, Baltimore, MD (US); THE REGENTS OF UNIVERSITY OF COLORADO, A BODY CORPORATE, Denver, CO (US)

(72) Inventors: Dax Fu, Short Hills, NJ (US); Chengfeng Merriman, Essex, MD (US); Liping Yu, Centennial, CO (US); Yong Gu, Centennial, CO (US)

(21) Appl. No.: 17/616,864

(22) PCT Filed: Jun. 8, 2020
(86) PCT No.: PCT/US2020/036625

§ 371 Date: Dec. 6, 2021

Related U.S. Application Data
(60) Provisional application No. 62/858,006, filed on Jun. 6, 2019.

(51) Int. Cl. G01N 33/564; G01N 33/49; G01N 33/533; G01N 33/536

(52) U.S. Cl. CPC G01N 33/564; G01N 33/49; G01N 33/533; G01N 33/536; G01N 2800/04

(57) ABSTRACT

The present invention relates to the field of autoimmunity. More specifically, the present invention provides compositions and methods useful for detecting autoantibodies. In one embodiment, a method for detecting autoantibodies to ZnT8 comprises the steps of (a) contacting in a first mixture a biological sample obtained from a patient with a ZnT8-antibody complex, wherein the ZnT8-antibody complex comprises ZnT8 and at least one detectably labeled anti-ZnT8 antibody or antigen-binding fragment thereof that specifically binds to the cytoplasmic domain of ZnT8; (b) contacting in a second mixture the first mixture of step (a) with an immunoglobulin G (IgG) labeled with a tag molecule; (c) contacting the second mixture of step (b) with a solid substrate coated with a capture molecule that specifically binds the tag molecule; and (d) detecting a signal emitted from the detectably labeled anti-ZnT8 antibody or antigen-binding fragment thereof.

### CROSS-REFERENCE TO RELATED APPLICATIONS

This application claims the benefit of U.S. Provisional Application No. 62/858,006, filed Jun. 6, 2019, which is incorporated herein by reference in its entirety.

### STATEMENT OF GOVERNMENTAL INTEREST

This invention was made with government support under grant no. GM065137, awarded by the National Institutes of Health. The government has certain rights in the invention.

### FIELD OF THE INVENTION

The present invention relates to the field of autoimmunity. More specifically, the present invention provides compositions and methods useful for detecting autoantibodies.

### INCORPORATION-BY-REFERENCE OF MATERIAL SUBMITTED ELECTRONICALLY

This application contains a sequence listing. It has been submitted electronically via EFS-Web as an ASCII text file entitled “P15404-02_ST25.txt.” The sequence listing is 74,689 bytes in size, and was created on Jun. 6, 2020. It is hereby incorporated by reference in its entirety.

### BACKGROUND OF THE INVENTION

The appearance of diabetes-associated autoantibodies is the first detectable sign of β-cell autoimmunity. At present, classification of autoimmune diabetes is based on the presence of autoantibodies (AAs) recognizing at least one of four major biochemical autoantigens: insulin, a 65 kD isoform of glutamic acid decarboxylase (GADA65), protein tyrosine phosphatase-related islet antigen-2 (IA2), and the C-terminal domain (CTD) of zinc transporter-8 (ZnT8) (1-3). Over the recent decades, the incidence of type-1 diabetes (T1D) has increased rapidly. This rise is characterized by a significantly increased prevalence of serum AAs to ZnT8 (ZnT8A) and IA2 (IA2A) in children and young adults with newly diagnosed T1D (4). IA2 is a single spanning membrane protein with a transmembrane anchor that contributes minimally to its AA-accessible protein surface (FIG. 1A). In contrast, ZnT8 contains a major transmembrane domain (TMD) twice the size of CTD (FIG. 1A). A significant portion of ZnT8 antigenicity is likely derived from TMD, but this domain is highly unstable in detergent solution, hence excluded from the standard AA assay based on detection of serum AAs to soluble autoantigens or soluble domains of membrane bound autoantigens. 60%-80% sera from patients with new onset T1D show positivity for anti-CTD AA (CTDA) (5). Recently, the present inventors developed a liposome-based method for purification of intact ZnT8 (TMD+CTD) (6). The purified ZnT8 in reconstituted proteoliposomes catalyzed vectorial zinc transport across the lipid bilayer, indicating that the TMD is properly folded and oriented in the membrane, exposing its extramembranous surfaces for antibody binding (FIG. 1A). A microarray of ZnT8 (TMD+CTD) proteoliposomes on a plasmonic gold chip (pGOLD) detected a significantly higher rate of AA positivity as compared with the CTD antigen alone, suggesting the presence of independent autoreactive epitopes on the extramembranous surface of TMD (7). However, a biochemical assay for unequivocal detection of serum AAs directed to TMD extramembranous surfaces (TMDA) is not available.

### SUMMARY OF THE INVENTION

The present invention is based, at least in part, on the development of an assay to detect autoantibodies targeting ZnT8 on the β-cell surface. Previous studies demonstrate ZnT8 is displayed on the β-cell surface upon insulin secretion and IgG from ZnT8A-positive T1D patient sera stains the surface of live pancreatic β-cells. To further identify autoantibodies (AAb) to the ZnT8 extracellular domain (ZnT8ec) and to develop a ZnT8ec AAb assay, the present inventors first stabilized its native structure without the N-terminal domain (a.a 66-369; R325 or W325) by forming ZnT8-intracellular domain Ab (ZnT8ic) complexes prior to release from liposome membranes. Sulfo-tagged ZnT8-Ab complexes were used as antigen in an electrochemiluminescence (ECL) AAb assay to analyze 130 sera (96 T1D patients, 22 T2D patients and 12 normal controls). Autoantibodies to ZnT8ec were identified in T1D patient sera by cross-reactivity of sera previously identified as exclusively ZnT8R or W positive, and ZnT8A negative sera. The prevalence of ZnT8ec AAbs in T1D patients was 21% (22/96:5/37 of R+ sera, 7/24 of W+ sera and 8/35 of ZnT8A- sera) compared to 1/22 T2D patients and 0/12 normal controls. These are the first biochemically defined AAbs from T1D sera reported to target the β-cell surface.

Accordingly, in one aspect, the present invention provides compositions and methods useful for detecting autoantibodies that bind to ZnT8. In particular embodiments, the autoantibodies bind to the extracellular domain of ZnT8. The compositions and methods can be used to diagnose or identify patients having T1D or a risk thereof. Indeed, the present invention represents an earlier detection method for T1D. The present invention can also be used to assess candidate T1D drugs. It is contemplated that other autoantibodies (for T1D) are detected in a multiplex manner with ZnT8A. For example, ZnT8A can be detected along with one or more of insulin autoantibody (IAA), glutamic acid decarboxylase autoantibody (GADA), and islet antigen 2 autoantibody (IA-2A).

In one embodiment, a method for detecting autoantibodies to ZnT8 comprises the steps of (a) contacting in a first mixture a biological sample obtained from a patient with a ZnT8-antibody complex, wherein the ZnT8-antibody complex comprises ZnT8 and at least one detectably labeled anti-ZnT8 antibody or antigen-binding fragment thereof that specifically binds to the cytoplasmic domain of ZnT8; (b) contacting in a second mixture the first mixture of step (a) with an immunoglobulin G (IgG) labeled with a tag molecule; (c) contacting the second mixture of step (b) with a solid substrate coated with a capture molecule that specifically binds the tag molecule; and (d) detecting a signal emitted from the detectably labeled anti-ZnT8 antibody or antigen-binding fragment thereof.

Alternatively, the patient sample can be contacted simultaneously with the ZnT8 complex and the tagged IgG. Thus, in a alternative embodiment, a method comprising the steps of (a) contacting in a mixture a biological sample obtained from a patient with (i) a ZnT8-antibody complex, wherein the ZnT8-antibody complex comprises ZnT8 and at least one detectably labeled anti-ZnT8 antibody or antigen-binding fragment thereof that specifically binds to the cytoplasmic domain of ZnT8, and (ii) an immunoglobulin G (IgG) labeled with a tag molecule; (b) contacting the mixture of step (a) with a solid substrate coated with a capture molecule that specifically binds the tag molecule; and (c) detecting a signal emitted from the detectably labeled anti-ZnT8 antibody or antigen-binding fragment thereof In certain embodiments, the biological sample is blood, plasma or serum.

In a specific embodiment, the at least one detectably labeled anti-ZnT8 antibody or antigen-binding fragment thereof comprises a Fab. In a more specific embodiment, the Fab comprises SEQ ID NO:32 and SEQ ID NO:37. In another embodiment, the Fab comprises (a) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions; and (b) light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions.

In an alternative embodiment, the Fab comprises SEQ ID NO:52 and SEQ ID NO:57. In another embodiment, the Fab comprises (a) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions; and (b) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

In particular embodiments, the at least one detectably labeled anti-ZnT8 antibody or antigen-binding fragment thereof comprises (a) a first Fab comprising SEQ ID NO:32 and SEQ ID NO:37; and a second Fab comprising SEQ ID NO:52 and SEQ ID NO:57. In another embodiment, the at least one detectably labeled anti-ZnT8 antibody or antigen-binding fragment thereof comprises (a) a first Fab comprising (i) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions, and (ii)light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions; and (b) a second Fab comprising (i) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions, and (ii) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

In particular embodiments, ZnT8 is full length ZnT8. In a specific embodiment, ZnT8 lacks an N-terminal domain. In a more specific embodiment, ZnT8 comprises amino acids 66-369 of SEQ ID NO:64. In particular embodiments, the detectable label is an electrochemiluminescent label. In a specific embodiment, the electrochemiluminescent label is a sulfo-tag. In certain embodiments, the cytoplasmic domain of ZnT8 comprises amino acids 276-369 of SEQ ID NO:64. In some embodiments, the tag molecule is biotin. In other embodiments, the capture molecule is streptavidin.

In another aspect, the present invention provides a composition comprising a ZnT8-antibody complex, and methods for making the same. In one embodiment, a ZnT8-antibody complex comprises (a) ZnT8; and (b) at least one anti-ZnT8 antibody or antigen-binding fragment thereof that specifically binds to the cytoplasmic domain of ZnT8. In one embodiment, ZnT8 is full length ZnT8. In another embodiment, ZnT8 lacks an N-terminal domain. In a more specific embodiment, ZnT8 comprises amino acids 66-369 of SEQ ID NO:64. In particular embodiments, the at least one anti-ZnT7 antibody or antigen-binding fragment thereof is detectably labeled. In one embodiment, the label is an ECL label. In a more specific embodiment, the ECL label is a sulfo-tag.

In certain embodiments, the at least one anti-ZnT8 antibody or antigen-binding fragment thereof comprises a Fab. In a specific embodiment, the Fab comprises SEQ ID NO:32 and SEQ ID NO:37. In another specific embodiment, the Fab comprises (a) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions; and (b) light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions.

In an alternative embodiment, the Fab comprises SEQ ID NO:52 and SEQ ID NO:57. In a specific embodiment, the Fab comprises (a) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions; and (b) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

In particular embodiments, the at least one detectably labeled anti-ZnT8 antibody or antigen-binding fragment thereof comprises (a) a first Fab comprising SEQ ID NO:32 and SEQ ID NO:37; and a second Fab comprising SEQ ID NO:52 and SEQ ID NO:57. In a specific embodiment, the at least one detectably labeled anti-ZnT8 antibody or antigen-binding fragment thereof comprises (a) a first Fab comprising (i) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions, and (ii) light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions; and (b) a second Fab comprising (i) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions, and (ii) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

### BRIEF DESCRIPTION OF THE FIGURES

FIG. 1A-1D. FIG. 1A: Structural models of membrane bound IA-2 and ZnT8 in relation to the cell surface membrane (grey balls and sticks). Magenta spheres are bound zinc ions in ZnT8. Two AAs marked as TMDA and CTDA bind to the extracellular surface of TMD and CTD, respectively. Proteins are drawn in 1:1 scale in yellow (IA-2), cyan (ZnT8), dark green (CTDA) or blue (TMDA). FIG. 1B: TMDA assay on ECL platform. ZnT8 was solubilized by detergent (grey balls and sticks) and stabilized by Fab (green bars) conjugated with a sulfo-tag (red star). FIG. 1C: Size-exclusion HPLC profile of purified ZnT8 (black) and ZnT8-Fab20 complex (red). FIG. 1D: Cryo-EM structure of a ZnT8-Fab20 complex with side view and top view from the cytosolic side. Green ribbons are fittings of the YiiP crystal structure to the electron density map.

FIG. 2A-2B. FIG. 2A: Scatter plot of TMDA level detected by ZnT8-Fab20 complex for 48 human sera from 33 T1D patients and 15 healthy controls. FIG. 2B: Linear correlation between ECL readout outs by ZnT8-Fab20 and ZnT8-Fab39 complex.

FIG. 3A-3B. FIG. 3A: CTD-ZnT8A radioimmunoassay. CTD-ZnT8A levels in each serum were measured against CTD-R and CTD-W variants. Magenta dashed lines indicate positivity cut-off. FIG. 3B: TMD-ZnT8A ECL assay. TMD-ZnT8A levels in each serum were measured against ZnT8-R and ZnT8-W variants in complex with Fab20 and Fab39. Note, ˜20% sera showed R/W cross-reactivity (diagonal datapoints).

FIG. 4. Prevalence of TMDA positivity in patients with T1D and T2D.

FIG. 5A-5B. FIG. 5A: Representative time course of AA levels over time in two DAISY cases. AA positivity cut-off is 1.0 for all AAs. FIG. 5B: Accumulative AA positivity from birth to clinical T1D. All children (n=10) were monitored for AA at 9 mo. Of age, then approximately in 3-mo intervals until the age of 2, and then 6-mo intervals afterwards. TMDA was the earliest AA to spear (median 1.2 yr), followed by IAA (median 2.1 yr), GADA (median 2.2 yr), IA-2A (median 2.6 yr), and the latest being CTDA (median 5.5 yr). Three AAs including IAA, GADA and IA-2A were measured by both RBA and ECL assays and the earliest age points of positive conversion from these two assays were selected for this plot.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

As used herein, the term “T1D” refers to type 1 diabetes.

As used herein, the term “ZnT8A” refers to a zinc transporter type 8 autoantibody.

The term “antibody” means an immunoglobulin molecule that recognizes and specifically binds to a target, such as a protein (e.g., the ZNT8, a subunit thereof, or the receptor complex), polypeptide, peptide, carbohydrate, polynucleotide, lipid, or combinations of the foregoing through at least one antigen recognition site within the variable region of the immunoglobulin molecule. A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region. The heavy chain constant region is comprised of three domains, CHI, CH2, and CH3. Each light chain is comprised of a “light chain variable region” or “light chain variable domain” (abbreviated herein as VL) and a light chain constant region. The light chain constant region is comprised of one domain, CI. The VH and VL regions can be further subdivided into regions of hypervariablity, termed Complementarity Determining Regions (CDR), interspersed with regions that are more conserved, termed framework regions (FRs). Each VH and VL region is composed of three CDRs and four FRs, arranged from amino-terminus to carboxy-terminus in the following order: FRI, CDRI, FR2, CDR2, FR3, CDR3, FR4. The variable regions of the heavy and light chains contain a binding domain that interacts with an antigen. As used herein, the term “antibody” encompasses intact polyclonal antibodies, intact monoclonal antibodies, antibody fragments (such as Fab, Fab', F(ab')2, Fd, Facb, and Fv fragments), single chain Fv (scFv), minibodies (e.g., sc(Fv)2, diabody), multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, detectable labels, radioisotopes, small molecule drugs, polypeptides, etc.

The term “isolated antibody” refers to an antibody that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody is purified (1) to greater than 95% by weight of antibody as determined by, for example, the Lowry method, and including more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, isolated antibody will be prepared by at least one purification step.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, “substantially purified” refers to being essentially free of other components. For example, a substantially purified polypeptide is a polypeptide which has been separated from other components with which it is normally associated in its naturally occurring state.

The term “humanized” immunoglobulin refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined herein, e.g., because the entire variable region of a chimeric antibody is non-human.

The term “antigen” is generally used in reference to any substance that is capable of reacting with an antibody. An antigen can also refer to a synthetic peptide, polypeptide, protein or fragment of a polypeptide or protein, or other molecule which elicits an antibody response in a subject, or is recognized and bound by an antibody.

The term “antigen-binding fragment” refers to a portion of an intact antibody and refers to the antigenic determining variable regions of an intact antibody. It is known in the art that the antigen-binding function of an antibody can be performed by fragments of a full-length antibody. Examples of antigen-binding antibody fragments include, but are not limited to Fab, Fab', F(ab')2, Facb, Fd, and Fv fragments, linear antibodies, single chain antibodies, and multi-specific antibodies formed from antibody fragments. In some instances, antibody fragments may be prepared by proteolytic digestion of intact or whole antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab')2 or Fab'; and plasmin digestion of whole antibodies yields Facb fragments.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced. The term “F(ab')2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced. The term “Fv” refers to an antibody fragment that consists of one NH and one N domain held together by noncovalent interactions.

The terms “ZNT8 antibody,” “anti-ZNT8 antibody,” “anti-ZNT8,” “antibody that binds to ZNT8” and any grammatical variations thereof refer to an antibody that is capable of specifically binding to ZNT8 with sufficient affinity such that the antibody is useful as a therapeutic agent or diagnostic reagent in targeting ZNT8. The extent of binding of an anti-ZNT8 antibody disclosed herein to an unrelated, non-ZNT8 protein is less than about 10% of the binding of the antibody to ZNT8 as measured, e.g., by a radioimmunoassay (RIA), BIACORE™ (using recombinant ZNT8 as the analyte and antibody as the ligand, or vice versa), or other binding assays known in the art. In certain embodiments, an antibody that binds to ZNT8 has a dissociation constant (KD) of <1 μM, <100 nM, <50 nM, <10 nM, or <1 nM.

The term “% identical” (“sequence identity”)between two polypeptide (or polynucleotide) sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site. Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2 (ClustalX is a version of the ClustalW2 program ported to the Windows environment). Another suitable program is MUSCLE. ClustalW2 and MUSCLE are alternatively available, e.g., from the European Bioinformatics Institute (EBI).

By “detectable label” is meant a composition that when linked to a molecule of interest renders the latter detectable via spectroscopic, photochemical, biochemical, immunochemical, chemical or electrochemiluminescent means. For example, detectable labels include radioactive isotopes, magnetic beads, metallic beads, colloidal particles, fluorescent dyes, electron-dense reagents, enzymes (for example, as commonly used in an ELISA), biotin, digoxigenin, or haptens. The labeling of an antigen can be carried out by any generally known method. Examples of the detectable label known to those skilled in the art include a fluorescent dye, an enzyme, a coenzyme, a chemiluminescent substance or a radioactive substance. Specific examples may include radioisotopes (³²P, ¹⁴C, ¹²⁵I, ³H, ¹³¹I and the like), fluorescein, rhodamine, dansyl chloride, umbelliferone, luciferase, peroxidase, alkaline phosphatase, beta-galactosidase, beta-glucosidase, horseradish peroxidase, glucoamylase, lysozyme, saccharide oxidase, microperoxidase, biotin and the like.

As used herein, the term “ECL” refers to electrochemiluminescence.

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of T1D. Moreover, a sample obtained from a patient can be divided and only a portion may be used for diagnosis. Further, the sample, or a portion thereof, can be stored under conditions to maintain sample for later analysis. In particular embodiments, the term sample includes blood and other liquid samples of biological origin (including, but not limited to, peripheral blood, serum, plasma, cerebrospinal fluid, urine, saliva, stool and synovial fluid). In a specific embodiment, the sample comprises blood. In another specific embodiment, the sample comprises serum. In yet another specific embodiment, the sample comprises plasma.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry. In certain embodiments, a sample comprises an optimal cutting temperature (OCT)-embedded frozen tissue sample.

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

The term “therapeutic agent” refers to any biological or chemical agent used in the treatment of a disease or disorder. Therapeutic agents include any suitable biologically active chemical compounds, biologically derived components such as cells, peptides, antibodies, and polynucleotides, and radiochemical therapeutic agents such as radioisotopes. In some embodiments, the therapeutic agent comprises a chemotherapeutic agent or an analgesic.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., an anti-ZnT8 antibody. The effect may be prophylactic in terms of completely or partially preventing a particular outcome, disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of preventing or treating any ZnT8-mediated disease including diabetes.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### II. Methods for Detecting ZnT8 Autoantibodies (ZnT8A)

In one aspect, the present invention provides methods for detecting ZnT8A. The methods comprise detecting ZnT8A using a ZnT8 antigen. The antigen can comprise full length ZnT8 or a fragment thereof In a specific embodiment, the ZnT8 lacks the N-terminal domain. In a more specific embodiment, the ZnT8 comprises amino acids 66-369 of SEQ ID NO:64.

In particular embodiments, ZnT8A are detected using a ZnT8-antibody complex. In certain embodiments, the complex comprises a ZnT8 antigen, for example, as described above. In further embodiments, the complex comprises at least one an anti-ZnT8 antibody or antigen-binding fragment thereof that specifically binds ZnT8. In a specific embodiment, the anti-ZnT8 antibody or antigen-binding fragment specifically binds the cytoplasmic domain (CTD) of ZnT8. In a more specific embodiment, the cytoplasmic domain of ZnT8 comprises amino acids 276-369 of SEQ ID NO:64. The use of at least one (at least 1, at least 2, at least 3, at least 4, and the like) anti-ZnT8 antibody or antigen-binding fragment thereof that specifically binds the cytoplasmic domain of ZnT8 blocks the binding of any autoantibodies to the cytoplasmic domain of ZnT8, thereby allowing detection of autoantibodies to the extracellular domain of ZnT8 that may be present in the patient sample.

In a specific embodiment, at least two anti-ZnT8 antibodies or antigen-binding fragments thereof are used to bind to the cytoplasmic domain of ZnT8. In one embodiment, the antibody or antigen-binding fragment thereof comprises a Fab. In particular embodiments, the Fab comprises the heavy chain and light chain of mAb12 (SEQ ID NOS:2 and 7, respectively), mAb16 (SEQ ID NOS:12 and 17, respectively), mAb17 (SEQ ID NOS:22 and 27, respectively), mAb20 (SEQ ID NOS:32 and 37, respectively), mAb28 (SEQ ID NOS:42 and 47, respectively) or mAb39 (SEQ ID NOS:52 and 57, respectively). In a specific embodiment, the antibody or antigen-binding fragment thereof comprises the heavy chain and light chain of mAb20 (SEQ ID NOS:32 and 37, respectively). In another specific embodiment, the antibody or antigen-binding fragment thereof comprises the heavy chain and light chain of mAb39 (SEQ ID NOS:52 and 57, respectively). In a more specific embodiment, the at least two antibodies or antigen-binding fragments thereof comprise the heavy chain and light chain of mAb20 (SEQ ID NOS:32 and 37, respectively) and the heavy chain and light chain of mAb39 (SEQ ID NOS:52 and 57, respectively).

In other embodiments, the Fab comprises heavy chain CDRs 1, 2 and 3 and light chain CDRs 1, 2 and 3. In particular embodiments, a Fab comprises the heavy chain CDRs 1, 2 and 3 and light chain CDRs 1, 2, and 3 of mAb 12 (SEQ ID NOS:3-5 and SEQ ID NOS:8-10, respectively), mAb 16 (SEQ ID NOS:13-15 and SEQ ID NOS:18-20, respectively), mAb 17 (SEQ ID NOS:23-25 and SEQ ID NOS:28-30, respectively), mAb 20 (SEQ ID NOS:33-35 and SEQ ID NOS:38-40, respectively), mAb 28 (SEQ ID NOS:43-45 and SEQ ID NOS:48-50, respectively), or mAb 39 (SEQ ID NOS:53-55 and SEQ ID NOS:58-60, respectively).

In alternative embodiments, the anti-ZnT8 antibody or antigen-binding fragment thereof comprises heavy chain CDRs 1, 2 and 3 and light chain CDRs 1, 2 and 3. In particular embodiments, the anti-ZnT8 antibody or antigen-binding fragment thereof comprises the heavy chain CDRs 1, 2 and 3 and light chain CDRs 1, 2, and 3 of mAb 12 (SEQ ID NOS:3-5 and SEQ ID NOS:8-10, respectively), mAb 16 (SEQ ID NOS:13-15 and SEQ ID NOS:18-20, respectively), mAb 17 (SEQ ID NOS:23-25 and SEQ ID NOS:28-30, respectively), mAb 20 (SEQ ID NOS:33-35 and SEQ ID NOS:38-40, respectively), mAb 28 (SEQ ID NOS:43-45 and SEQ ID NOS:48-50, respectively), or mAb 39 (SEQ ID NOS:53-55 and SEQ ID NOS:58-60, respectively).

In a specific embodiment, at least two anti-ZnT8 antibodies or antigen-binding fragments thereof are used to bind to the cytoplasmic domain of ZnT8. Each of the at least two anti-ZnT8 antibodies or antigen-binding fragments thereof comprises heavy chain CDRs 1, 2 and 3 and light chain CDRs 1, 2 and 3. In particular embodiments, a first anti-ZnT8 antibody or antigen-binding fragment thereof comprises the heavy chain CDRs 1, 2 and 3 and light chain CDRs 1, 2, and 3 of mAb 20 (SEQ ID NOS:33-35 and SEQ ID NOS:38-40, respectively), and a second anti-ZnT8 antibody or antigen-binding fragment thereof comprises the heavy chain CDRs 1, 2 and 3 and light chain CDRs 1, 2, and 3 of mAb 39 (SEQ ID NOS:53-55 and SEQ ID NOS:58-60, respectively).

In further embodiments, the anti-ZnT8 antibody or antigen-binding fragment is detectably labeled. In certain embodiments, the detectable label comprises an electrochemiluminescence (ECL) label. In other embodiments, the detectable label comprises an enzyme including, but not limited to, luciferase, sulfatase, phosphatase (e.g., alkaline phosphatase), beta-galactosidase, glucoamylase, beta-glucosidase, lysozyme, saccharide oxidase, microperoxidase, and/or peroxidase (e.g., horseradish peroxidase). In another embodiment, the detectable label comprises a fluorogen. In a further embodiment, the detectable label comprises a nucleotide sequence. In other embodiments, the detectable label comprises a radioactive isotope (such as, but not limited to, ³²P, ¹⁴C, ¹²⁵I, ³H, ¹³¹I and the like), magnetic bead, metallic bead, colloidal particle, fluorescent dye, electron-dense reagent, chemiluminescent dye, enzyme, co-enzyme, biotin, digoxigenin, and/or hapten. Non-limiting examples of dyes include fluorescein, rhodamine, dansyl chloride, and umbelliferone.

In particular embodiments, the detectable label comprises an ECL label. Preferred ECL labels include luminescent organometallic complexes of Ru, Os and Re. Some especially useful materials are polypyridyl complexes of ruthenium and osmium, in particular, complexes having the structure ML¹L²L³ where M is ruthenium or osmium, and L¹,L²and L³ each are bipyridine, phenathroline, substituted bipyridine and/or substituted phenanthroline. In specific embodiments, the ECL label comprises a ruthenium complex. In more specific embodiments, the ECL label comprises ruthenium-tris-bipyridine. In a specific embodiment, the ECL label comprises [Ru(BPy)₃]²⁺. In another embodiment, the ECL label comprises a sulfo-tag.

In one embodiment, a method comprises the steps of (a) contacting in a first mixture a biological sample obtained from a patient with a ZnT8-antibody complex, wherein the ZnT8-antibody complex comprises ZnT8 and at least one detectably labeled anti-ZnT8 antibody or antigen-binding fragment thereof that specifically binds to the cytoplasmic domain of ZnT8; (b) contacting in a second mixture the first mixture of step (a) with an immunoglobulin G (IgG) labeled with a tag molecule; (c) contacting the second mixture of step (b) with a solid substrate coated with a capture molecule that specifically binds the tag molecule; and (d) detecting a signal emitted from the detectably labeled anti-ZnT8 antibody or antigen-binding fragement thereof.

Alternatively, the patient sample can be contacted simultaneously with the ZnT8 complex and the tagged IgG. Thus, in a alternative embodiment, a method comprises the steps of (a) contacting in a mixture a biological sample obtained from a patient with (i) a ZnT8-antibody complex, wherein the ZnT8-antibody complex comprises ZnT8 and at least one detectably labeled anti-ZnT8 antibody or antigen-binding fragment thereof that specifically binds to the cytoplasmic domain of ZnT8, and (ii) an immunoglobulin G (IgG) labeled with a tag molecule; (b) contacting the mixture of step (a) with a solid substrate coated with a capture molecule that specifically binds the tag molecule; and (c) detecting a signal emitted from the detectably labeled anti-ZnT8 antibody or antigen-binding fragment thereof

In certain embodiments, the sample comprises a biological sample from a mammal. In other embodiments, the biological sample comprises blood, serum, plasma, urine and/or saliva from the mammal. In a specific embodiment, the sample comprises blood. In another specific embodiment, the sample comprises serum. In yet another specific embodiment, the sample comprises plasma.

Any appropriate tag molecule can be used providing it facilitates the binding of the tagged IgG to its corresponding partner capture molecule. Exemplary tags can be c-myc tags (which can be captured via an anti c-myc antibody), His tags (which can for example be capture via a nickel surface), biotin (which can be captured via streptavidin molecules), or a phage surface protein such as gp8 (which can be captured via an anti-gp8 antibody), or antigen peptide tags such as FLAG or HA, which may be recognized by an antibody. Such tag can be directly or indirectly labeled. In certain embodiments, the tag molecule comprises biotin and the capture molecule comprises streptavidin.

In other embodiments, the methods of the present invention further comprise detecting other autoantibodies associated with T1D including insulin autoantibody (IAA), glutamic acid decarboxylase autoantibody (GADA), and/or islet antigen 2 autoantibody (IA-2A). In certain embodiments, the detected autoantibodies comprise ZnT8A and at least one selected from the group consisting of IAA, GADA, and IA-2A.

The antigens to the other autoantibodies can be labeled with the same or different detectable label. In other embodimnents, the methods can utilize an antigen comprising a detectable label and antigen comprising a tag molecule. It is contemplated that, in certain embodiments, the autoantibody specific to the antigen would bridge antigen comprising a detectable label and antigen comprising a tag molecule, forming a detectably labeled antigen-autoantibody-tagged antigen complex. The solid substrate would comprise a capture molecule that is specific for the tag molecule of the particular antigen. In certain embodiments, the solid substrate comprises a plurality of non-overlapping areas, wherein each non-overlapping area comprises a capture molecule that binds specifically to a tag molecule.

### III. Anti-ZnT8 Antibodies

The antibodies or antigen-binding fragment thereof of this disclosure specifically bind to ZNT8. In specific embodiments, these antibodies or antigen-binding fragments specifically bind to human ZNT8. “Specifically binds” as used herein means that the antibody or antigen-binding fragment preferentially binds ZNT8 (e.g., human ZNT8, mouse ZNT8) over other proteins. In certain instances, the anti-ZNT8 antibodies of the disclosure have a higher affinity for ZNT8 than for other proteins. Anti-ZNT8 antibodies that specifically bind ZNT8 may have a binding affinity for human ZNT8 of less than or equal to 1×10−7 M, less than or equal to 2×10−7 M, less than or equal to 3×10−7 M, less than or equal to 4×10−7 M, less than or equal to 5×10−7 M, less than or equal to 6×10−7 M, less than or equal to 7×10−7 M, less than or equal to 8×10−7 M, less than or equal to 9×10−7 M, less than or equal to 1×10−8 M, less than or equal to 2×10−8 M, less than or equal to 3×10−8 M, less than or equal to 4×10−8 M, less than or equal to 5 ×10−8 M, less than or equal to 6×10−8 M, less than or equal to 7×10−8 M, less than or equal to 8×10−8 M, less than or equal to 9×10−8 M, less than or equal to 1×10−9 M, less than or equal to 2×10−9 M, less than or equal to 3×10−9 M, less than or equal to 4×10−9 M, less than or equal to 5×10−9 M, less than or equal to 6×10−9 M, less than or equal to 7×10−9 M, less than or equal to 8×10−9 M, less than or equal to 9×10−9 M, less than or equal to 1×10−10 M, less than or equal to 2×10−10 M, less than or equal to 3×10−10 M, less than or equal to 4×10−10 M, less than or equal to 5×10−10 M, less than or equal to 6×10−10 M, less than or equal to 7×10−10 M, less than or equal to 8×10−10 M, less than or equal to 9×10−10 M, less than or equal to 1×10−11 M, less than or equal to 2×10−11 M, less than or equal to 3×10−11 M, less than or equal to 4×10−11 M, less than or equal to 5×10−11 M, less than or equal to 6×10−11 M, less than or equal to 7×10−11 M, less than or equal to 8×10−11 M, less than or equal to 9×10−11 M, less than or equal to 1×10−12 M, less than or equal to 2×10−12 M, less than or equal to 3×10−12 M, less than or equal to 4×10−12 M, less than or equal to 5×10−12 M, less than or equal to 6×10−12 M, less than or equal to 7×10−12 M, less than or equal to 8×10−12 M, or less than or equal to 9×10−12 M. Methods of measuring the binding affinity of an antibody are well known in the art and include Surface Plasmon Resonance (SPR) (Morton and Myszka “Kinetic analysis of macromolecular interactions using surface plasmon resonance biosensors” Methods in Enzymology (1998) 295, 268-294), Bio-Layer Interferometry, (Abdiche et al “Determining Kinetics and Affinities of Protein Interactions Using a Parallel Real-time Label-free Biosensor, the Octet” Analytical Biochemistry (2008) 377, 209-217), Kinetic Exclusion Assay (KinExA) (Darling and Brault “Kinetic exclusion assay technology: characterization of molecular interactions” Assay and Drug Dev Tech (2004) 2, 647-657), isothermal calorimetry (Pierce et al “Isothermal Titration Calorimetry of Protein-Protein Interactions” Methods (1999) 19, 213-221) and analytical ultracentrifugation (Lebowitz et al “Modem analytical ultracentrifugation in protein science: A tutorial review” Protein Science (2002), 11:2067-2079).

### A. Antibody Fragments

The present disclosure encompasses the antibody fragments or domains described herein that retains the ability to specifically bind to ZNT8 (e.g., human ZNT8). Antibody fragments include, e.g., Fab, Fab', F(ab')2, Facb, and Fv. These fragments may be humanized or fully human. Antibody fragments may be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab')2 or Fab'; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J Immunol., 152:2968-2976 (1994); Better, M. and Horwitz, A. H., Methods in Enzymology, 178:476-496 (1989); Pluckthun, A and Skerra, A, Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab'-SH fragments can be directly recovered from E. coli and chemically coupled to form F(ab)2 fragments (Carter et al., Bio/Technology, 10:163- 167 (1992)). According to another approach, F(ab')2 fragments can be isolated directly from recombinant host cell culture. Fab and F(ab') 2 fragment with increased in vivo half-life comprising a salvage receptor binding epitope residues are described in U.S. Pat. No. 5,869,046.

### B. Minibodies

Also encompassed are minibodies of the antibodies described herein. Minibodies of anti-ZNT8 antibodies include diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. U.S.A., 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U.S.A., 85:5879-5883 (1988); and Pluckthun, “The Pharmacology of Monoclonal Antibodies” Vol.113, Ed Resenburg and Moore, Springer Verlag, New York, pp.269-315, (1994)). Each variable domain (or a portion thereof) is derived from the same or different antibodies. Single chain Fv molecules preferably comprise an scFv linker interposed between the VH domain and the VL domain. Exemplary scFv molecules are known in the art and are described, for example, in U.S. Pat. No. 5,892,019; Ho et al, Gene, 77:51 (1989); Bird et al., Science, 242:423 (1988); Pantoliano et al, Biochemistry, 30: 101 17 (1991); Milenic et al, Cancer Research, 51 :6363 (1991); Takkinen et al, Protein Engineering, 4:837 (1991).

The term “scFv linker” as used herein refers to a moiety interposed between the VL and VH domains of the scFv. The scFv linkers preferably maintain the scFv molecule in an antigen-binding conformation. In one embodiment, an scFv linker comprises or consists of an scFv linker peptide. In certain embodiments, an scFv linker peptide comprises or consists of a Gly-Ser peptide linker. In other embodiments, an scFv linker comprises a disulfide bond.

The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-ZNT8 antibody or antigen-binding fragment thereof described herein.

An sc(Fv)2 is a minibody in which two VHs and two VLs are linked by a linker to form a single chain (Hudson, et al., J Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however, the order of the two VHs and two VLs is not limited to the above arrangement, and they may be arranged in any order. Examples of arrangements are listed below:
[VL] linker [VH] linker [VH] linker [VL]
[VH] linker [VL] linker [VL] linker [VH]
[VH] linker [VH] linker [VL] linker [VL]
[VL] linker [VL] linker [VH] linker [VH]
[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about 3 to 25 residues (e.g., 5 , 6, 7, 8, 9, 10, 11, 12, 13, 14, 1S, 16, 17, 18) can be used as a linker.

In other embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidy Ipropionate) (DSP), dithiobis(sulfosuccinimidy Ipropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the antibody fragments or minibodies may include modifications such as substitutions, deletions, additions, and/or insertions. For example, the modification may be in one or more of the CDRs of the anti-ZNT8 antibodies described herein. In certain embodiments, the modification involves one, two, or three amino acid substitutions in one, two, or three CDRs of the VH and/or one, two, or three CDRs of the VL domain of the anti-ZNT8 minibody. Such substitutions are made to improve the binding and/or functional activity of the anti-ZNT8 minibody. In other embodiments, one, two, or three amino acids of one or more of the six CDRs of the anti- ZNT8 antibody or antigen-binding fragment thereof may be deleted or added as long as there is ZNT8 binding and/or functional activity when VH and VL are associated.

### C. VHH

VHH also known as nanobodies are derived from the antigen-binding variable heavy chain regions (VHHs) of heavy chain antibodies found in camels and llamas, which lack light chains. The present disclosure encompasses VHHs that specifically bind ZNT8.

### D. Variable Domain of New Antigen Receptors (VNARs)

A VNAR is a variable domain of a new antigen receptor (IgNAR). IgNARs exist in the sera of sharks as a covalently linked heavy chain homodimer. It exists as a soluble and receptor bound form consisting of a variable domain (VNAR) with differing numbers of constant domains. The VNAR is composed of a CDR1 and CDR3 and in lieu of a CDR2 has HV2 and HV4 domains (see, e.g., Barelle and Porter, Antibodies, 4:240-258 (2015)). The present disclosure encompasses VNARs that specifically bind ZNT8.

### E. Constant Regions

Antibodies of this disclosure can be whole antibodies or single chain Fc (scFc) and can comprise any constant region known in the art. The light chain constant region can be, for example, a kappa- or lambda-type light chain constant region, e.g., a human kappa or human lambda light chain constant region. The heavy chain constant region can be, e.g., an alpha-, delta-, epsilon-, gamma-, or mu-type heavy chain constant region, e.g., a human alpha-, human delta-, human epsilon-, human gamma-, or human mu-type heavy chain constant region. In certain instances, the anti-ZNT8 antibody is an IgA antibody, an IgD antibody, an IgE antibody, an IgG1 antibody, an IgG2 antibody, an IgG3 antibody, an IgG4 antibody, or an IgM antibody.

In one embodiment, the light or heavy chain constant region is a fragment, derivative, variant, or mutein of a naturally occurring constant region. In some embodiments, the variable heavy chain of the anti-ZNT8 antibodies described herein is linked to a heavy chain constant region comprising a CH1 domain and a hinge region. In some embodiments, the variable heavy chain is linked to a heavy chain constant region comprising a CH2 domain. In some embodiments, the variable heavy chain is linked to a heavy chain constant region comprising a CH3 domain. In some embodiments, the variable heavy chain is linked to a heavy chain constant region comprising a CH2 and CH3 domain. In some embodiments, the variable heavy chain is linked to a heavy chain constant region comprising a hinge region, a CH2 and a CH3 domain. The CH1, hinge region, CH2, and/or CH3 can be from an IgG antibody (e.g., IgGI, IgG4). In certain embodiments, the variable heavy chain of an anti-ZNT8 antibody described herein is linked to a heavy chain constant region comprising a CHI domain, hinge region, and CH2 domain from IgG4 and a CH3 domain from IgGI. In certain embodiments such a chimeric antibody may contain one or more additional mutations in the heavy chain constant region that increase the stability of the chimeric antibody. In certain embodiments, the heavy chain constant region includes substitutions that modify the properties of the antibody.

In certain embodiments, an anti-ZNT8 antibody of this disclosure is an IgG isotype antibody. In one embodiment, the antibody is IgG1. In another embodiment, the antibody is IgG2. In yet another embodiment, the antibody is IgG4. In some instances, the IgG4 antibody has one or more mutations that reduce or prevent it adopting a functionally monovalent format. For example, the hinge region of IgG4 can be mutated to make it identical in amino acid sequence to the hinge region of human IgG1 (mutation of a serine in human IgG4 hinge to a proline). In some embodiments, the antibody has a chimeric heavy chain constant region (e.g., having the CH1, hinge, and CH2 regions of IgG4 and CH3 region of IgG1).

### F. Bispecific Antibodies

In certain embodiments, an anti-ZNT8 antibody of this disclosure is a bispecific antibody. Bispecific antibodies are antibodies that have binding specificities for at least two different epitopes. Exemplary bispecific antibodies may bind to two different epitopes of the ZNT8 protein. Other such antibodies may combine a ZNT8 binding site with a binding site for another protein. Bispecific antibodies can be prepared as full length antibodies or low molecular weight forms thereof (e.g., F(ab') 2 bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### G. Conjugated Antibodies

The antibodies or antigen-binding fragments disclosed herein may be conjugated to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), human serum albumin or a fragment thereof, radioactive materials (e.g., 90Y, 131I), fluorescent substances, luminescent substances, haptens, enzymes, metal chelates, detectable labels and drugs.

In certain embodiments, an anti-ZNT8 antibody or antigen-binding fragment thereof is modified with a moiety that improves its stabilization and/or retention in circulation, e.g., in blood, serum, or other tissues, e.g., by at least 1.5, 2, 5, 10, 15, 20, 25, 30, 40, or 50 fold. For example, the anti-ZNT8 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-ZNT8 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies or fragments can be prepared by performing chemical modifications on the antibodies or the lower molecular weight forms thereof described herein. Methods for modifying antibodies are well known in the art.

### IV. Characterization of Antibodies

The ZNT8 binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-ZNT8 antibody or functional fragment thereof) and a target (e.g., ZNT8) can be analyzed using the OCTET® systems. In this method, one of several variations of instruments (e.g., OCTET® QKe and QK), made by the ForteBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-ZNT8 antibody or functional fragment thereof) and a target (e.g., ZNT8) can be analyzed using Surface Plasmon Resonance (SPR). SPR or Biomolecular Interaction Analysis (BIA) detects biospecific interactions in real time, without labeling any of the interactants.

Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which is measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem 63:2338-2345; Szabo et al. (1995) Curr. Opin. Struct. Biol. 5:699-705 and on-line resources provide by BlAcore International AB (Uppsala, Sweden). Information from SPR can be used to provide an accurate and quantitative measure of the equilibrium dissociation constant (Kd), and kinetic parameters, including Kon and Koff, for the binding of a biomolecule to a target.

Epitopes can also be directly mapped by assessing the ability of different anti-ZNT8 antibodies or functional fragments thereof to compete with each other for binding to human ZNT8 using BIACORE chromatographic techniques (Pharmacia BlAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen-binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

### V. Affinity Maturation

In one embodiment, an anti-ZNT8 antibody or antigen-binding fragment thereof is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used. Methods of effecting affinity maturation include random mutagenesis (e.g., Fukuda et al., Nucleic Acids Res., 34:e127 (2006); targeted mutagenesis (e.g., Rajpal et al., Proc. Natl. Acad. Sci. USA, 102:8466-71 (2005); shuffling approaches (e.g., Jermutus et al., Proc. Natl. Acad. Sci. USA, 98:75-80 (2001); and in silica approaches (e.g., Lippow et al., Nat. Biotechnol., 25: 1171-6 (2005).

In some embodiments, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criterion for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (198 1) Anal. Biochem., 118:131; and Thotakura et al. (1987) Meth. Enzymol., 138:350). See, e.g., U.S. Pat. No. 5,869,046 for a modification that increases in vivo half-life by providing a salvage receptor binding epitope.

In one embodiment, an anti-ZNT8 antibody has one or more CDR sequences (e.g., a Chothia, an enhanced Chothia, or Kabat CDR) that differ from those described herein. In one embodiment, an anti-ZNT8 antibody has one or more CDR sequences include amino acid changes, such as substitutions of 1, 2, 3, or 4 amino acids if a CDR is 5-7 amino acids in length, or substitutions of 1, 2, 3, 4, or 5, of amino acids in the sequence of a CDR if a CDR is 8 amino acids or greater in length. The amino acid that is substituted can have similar charge, hydrophobicity, or stereochemical characteristics. In some embodiments, the amino acid substitution(s) is a conservative substitution. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In other embodiments, the amino acid substitution(s) is a non-conservative substitution. The antibody or antibody fragments thereof that contain the substituted CDRs can be screened to identify antibodies of interest.

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J Immun., 147:26S7-62 (1991); Morgan et al.,Immunology, 86:319-24 (199S)), or changing the species from which the constant region is derived.

### VI. Methods of Producing Anti-ZNT8 Antibodies

The anti-ZNT8 antibodies (or antigen-binding domain(s) of an antibody or functional fragment thereof) of this disclosure may be produced in bacterial or eukaryotic cells. To produce the polypeptide of interest, a polynucleotide encoding the polypeptide is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HB1O1, or XL I-Blue is used as a host, the vector must have a promoter, for example, a lacZ promoter (Ward et al., 341:544-546 (1989), araB promoter (Better et al., Science, 240: 1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, 293, 293T, and NIH3T3 cells, the expression vector includes a promoter necessary for expression in these cells, for example, an SV40 promoter (Mulligan et al., Nature, 277:108 (1979)), MMLV-LTR promoter, EF la promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins ofreplication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, the antibodies are produced in mammalian cells. Exemplary mammalian host cells for expressing a polypeptide include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

The antibodies of the present disclosure can be isolated from inside or outside (such as medium) of the host cell and purified as substantially pure and homogenous antibodies. Methods for isolation and purification commonly used for polypeptides may be used for the isolation and purification of antibodies described herein, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

The present disclosure also provides a nucleic acid molecule or a set of nucleic acid molecules encoding an anti-ZNT8 antibody or antigen-binding molecule thereof disclosed herein. In one embodiment, the invention includes a nucleic acid molecule encoding a polypeptide chain, which comprises a light chain of an anti- ZNT8 antibody or antigen-binding molecule thereof as described herein. In one embodiment, the invention includes a nucleic acid molecule encoding a polypeptide chain, which comprises a heavy chain of an anti-ZNT8 antibody or antigen-binding molecule thereof as described herein.

Also provided are a vector or a set of vectors comprising such nucleic acid molecule or the set of the nucleic acid molecules or a complement thereof, as well as a host cell comprising the vector.

The instant disclosure also provides a method for producing a ZNT8 or antigen-binding molecule thereof or chimeric molecule disclosed herein, such method comprising culturing the host cell disclosed herein and recovering the antibody, antigen-binding molecule thereof, or the chimeric molecule from the culture medium.

A variety of methods are available for recombinantly producing a ZNT8 antibody or antigen-binding molecule thereof disclosed herein, or a chimeric molecule disclosed herein. It will be understood that because of the degeneracy of the code, a variety of nucleic acid sequences will encode the amino acid sequence of the polypeptide. The desired polynucleotide can be produced by de novo solid-phase DNA synthesis or by PCR mutagenesis of an earlier prepared polynucleotide.

For recombinant production, a polynucleotide sequence encoding a polypeptide (e.g., a ZNT8 antibody or antigen-binding molecule thereof disclosed herein, or any of the chimeric molecules disclosed herein) is inserted into an appropriate expression vehicle, i.e., a vector which contains the necessary elements for the transcription and translation of the inserted coding sequence, or in the case of an RNA viral vector, the necessary elements for replication and translation.

The nucleic acid encoding the polypeptide (e.g., a ZNT8 antibody or antigen-binding molecule thereof disclosed herein, or any of the chimeric molecules disclosed herein) is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14:725) and electroporation (Neumann et al. 1982, EMBO J. 1:841). A variety of host- expression vector systems can be utilized to express the polypeptides described herein (e.g., a ZNT8 antibody or antigen-binding molecule thereof disclosed herein, or any of the chimeric molecules disclosed herein) in eukaryotic cells. In one embodiment, the eukaryotic cell is an animal cell, including mammalian cells (e.g., 293 cells, PerC6, CHO, BHK, Cos, HeLa cells). When the polypeptide is expressed in a eukaryotic cell, the DNA encoding the polypeptide (e.g., a ZNT8 antibody or antigen-binding molecule thereof disclosed herein, or any of the chimeric molecules disclosed herein) can also code for a signal sequence that will permit the polypeptide to be secreted. One skilled in the art will understand that while the polypeptide is translated, the signal sequence is cleaved by the cell to form the mature chimeric molecule. Various signal sequences are known in the art and familiar to the skilled practitioner. Alternatively, where a signal sequence is not included, the polypeptide (e.g., a ZNT8 antibody or antigen- binding molecule thereof disclosed herein, or any of the chimeric molecules disclosed herein) can be recovered by lysing the cells.

### VII. Kits

The compositions of the present invention can be provided in a kit. In one embodiment, a kit comprises a solid substrate for capturing the ZnT8A bound to the ZnT8-antibody complex. In certain embodiments, the solid substrate comprises a silicon wafer, glass, metal, plastic, ceramic, metal alloy, polymer or any combinations thereof. In a particular embodiment, the solid substrate comprises a plate. In a more particular embodiment, the plate is a 96-well plate from, for example, Meso Scale Diagnostics, LLC (Rockville, MD).

The kit can further comprise immunoglobulin G (IgG). In further embodiments, the kit comprises a tag molecule for tagging IgG including, for example, biotin. In other embodiments, the kit comprises a capture molecule including, for example, streptavidin. In other embodiments, the kit comprises ZnT8 antigen including, for example, full length ZnT8 or a fragment thereof. The kits of the present invention can also comprise an anti-ZnT8 antibody or antigen-binding fragment thereof. In other embodiments, the kit comprises a detectable label including, for example, an ECL label.

In addition to the anti-ZNT8 antibody or fragment thereof, the kit can include other ingredients, such as a solvent or buffer, a stabilizer, or a preservative, performing the assay. The anti-ZNT8 antibody or fragment thereof can be provided in any form, e.g., liquid, dried or lyophilized form, preferably substantially pure and/or sterile. When the agents are provided in a liquid solution, the liquid solution preferably is an aqueous solution. When the anti-ZNT8 antibody or fragment thereof is provided as a lyophilized product, the lyophilized powder is generally reconstituted by the addition of a suitable solvent. The solvent, e.g., sterile water or buffer (e.g., PBS), can optionally be provided in the kit.

The kit can include one or more containers for the composition or compositions containing the agents. In some embodiments, the kit contains separate containers, dividers or compartments for the components and for any informational material. For example, the components can be contained in a bottle, vial, or tube, and the informational material can be contained in a plastic sleeve or packet. In other embodiments, the separate elements of the kit are contained within a single, undivided container. For example, a component can be contained in a bottle, vial or tube that has attached thereto the informational material in the form of a label. The containers of the kits can be air tight, waterproof (e.g., impermeable to changes in moisture or evaporation), and/or light-tight (e.g., in the case of an ECL label).

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Identification of Autoantibodies to Extramembranous Epitopes of ZnT8 in Patients with T1D

Among the four major autoantigens, the expression of ZnT8 and insulin are islet-specific while GAD65 and IA2 are broadly distributed in neuroendocrine cells (8). In (β-cells, ZnT8 has a primary function as a zinc sequestrating transporter in the insulin secretory granules (9). This granular membrane protein is also abundantly displayed on the cell surface following glucose-stimulated insulin secretion (GSIS) (10). The dynamics of ZnT8 subcellular locations may promote intramolecular epitope spreading from an initial set of TMD epitopes on the extracellular surface of live (β-cells to intracellular CTD epitopes as a result of secondary apoptotic exposure (11). For childhood diabetes, especially in very young children, IAA is usually recognized as the first islet AA appearing in the natural history of T1D development, GADA for the second, and IA-2A and ZnT8A are usually considered as later markers closer to clinical disease (12,13). However, almost all ZnT8A data in the literature by default indicate autoimmune reactivity toward CTD. Recently, the present inventors identified a subclass of serum ZnT8As directed to surfaced ZnT8 on live (β-cells (14), raising the possibility that TMDA may precede the occurrence of CTDA arose from epitope spreading (15). At present, the prevalence of TMDA in patients with T1D, and the temporal relationship between the first appearance of TMDA and other AAs are unclear.

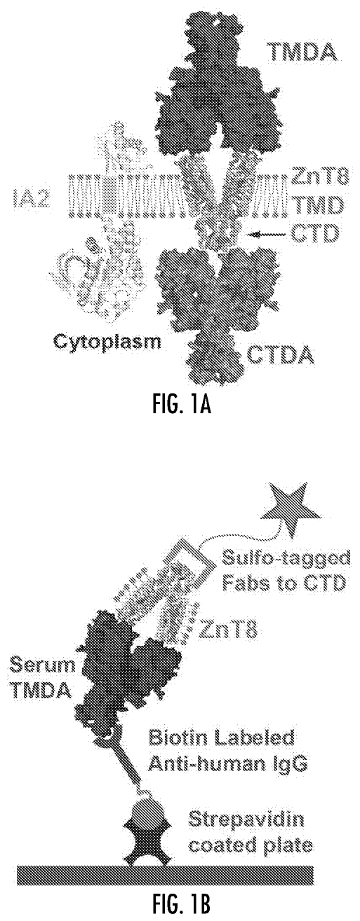

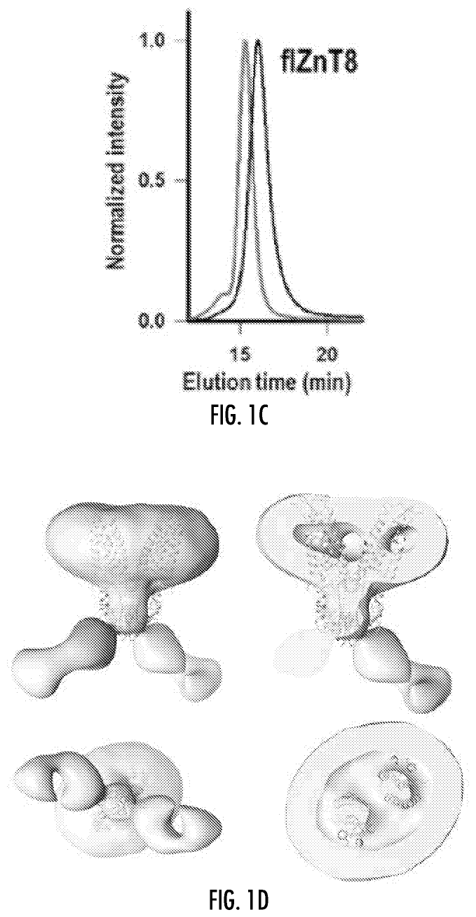

To detect serum AA binding to extramembranous epitopes on TMD, the present inventors developed a ZnT8-antibody complex for AA detection on an electrochemiluminescence (ECL) platform (16). In this ECL assay, two high affinity antigen-binding fragments (Fabs) of monoclonal anti-ZnT8 antibodies (mAb20 and mAb39) were used to stabilize an intact ZnT8 antigen in detergent solution (FIG. 1B). The Fab-ZnT8 complex preferentially exposes the extracellular surface of TMD to TMDA binding, leading to a positive ECL readout through a sulfo-tag covalently linked to bound Fabs (FIG. 1B). The present inventors validated the TMDA assay by correlation analysis of two nonsynonymous polymorphic variants of ZnT8, and applied the assay to evaluate the prevalence of TMDA in different cohorts of diabetic patients and healthy controls. The present inventors are performing a longitudinal follow-up of the first appearance of TMDA from birth to clinical T1D in comparison to the first appearance of other islet AAs. It is expected that TMDA is an earlier AA independent of CTDA, thereby providing direct biochemical evidence for a prevalent presence of surface-targeted TMDAs during T1D progression.

### Results

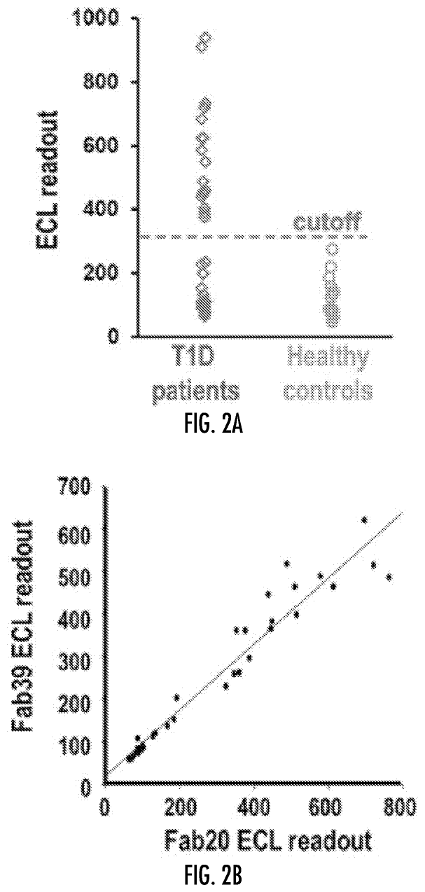

Assay design. The present inventors adapted the detergent solubilized ZnT8 antigen to an ECL platform with modifications for detecting antibody-antigen complexes (16). Fab20 and Fab39 formed stable binary complexes with ZnT8 as well as a Fab20-ZnT8-Fab39 ternary complex as showed by analytical sizing HPLC (FIG. 1C). The complex peaks remained monodisperse in diluted solution (1 μg/ml) with 2× molar excess of free Fabs to maintain a full Fab binding occupancy for multiple days. Fab20-ZnT8 complex was concentrated to >3 mg/ml without appreciable protein aggregation. The concentrated protein was subjected to cryo-EM single particle analysis, yielding a 3D-recogntition of ZnT8-Fab20 binding complex at 20-angstrom resolution (FIG. 1D). Fitting the electron density map with a ZnT8 homology model based on the crystal structure of YiiP indicated that human ZnT8 adopted a characteristic dimeric conformation in which two TMDs are splayed open and two CTDs form the stem of a Y-architecture (17). Two Fabs were clearly visible in association with each of two CTDs in a ZnT8 homodimer. An earlier Fab20-ZnT8-Fab39 ternary structure also mapped Fab39 binding to CTD to a distinct epitope in an orientation approximately 90-degree relative to that of Fab20 binding (18). Thus, the solvent accessible surface of CTD is completely shielded from additional CTDA access in a Fab20-ZnT8-Fab39 ternary complex. The transmembrane sector of TMD was fully embedded in the detergent micelles whereas the extracellular surface of TMD is wide open to potential TMDA binding. By comparison, the cytosolic surface of TMD was rather limited and partially obstructed from TMDA binding by neighboring Fab20 in the binding complex (FIG. 1D). Hence, ZnT8-Fab complexes by design are expected to preferentially detect TMDA directed to the extracellular surface, although detection of TMDA to the cytosolic surface cannot be ruled out at this time. The TMDA assay was performed in solution by incubating human sera with ZnT8-Fab complex, followed by adding biotin labeled anti-human IgG to capture TMDA-ZnT8-Fab complexes on a streptavidin coated plate for ECL readout (FIG. 2B).

Assay calibration. Applying this assay to 33 samples from T1D patients and 15 samples from healthy controls, 17/33 of patients with T1D were found positive for TMD-ZnT8A based on a positivity cut-off set to the 100th percentile of healthy controls (0/15) (FIG. 2A). The ECL assay signals were very similar when either ZnT8-Fab20 or ZnT8-Fab39 complex was used to detect serum TMD-ZnT8As (FIG. 2B). The prevalence of TMD-ZnT8A positivity suggested by the ECL assay was much higher than that estimated by the pGOLD assay (52%/17%). Since polyclonal serum ZnT8As were likely composed of many different TMDAs and CTDAs, there was a possibility that certain serum CTDAs with ultra-high affinity could displace Fab20/Fab30 binding to CTD, giving rise to false positive for TMDAs.

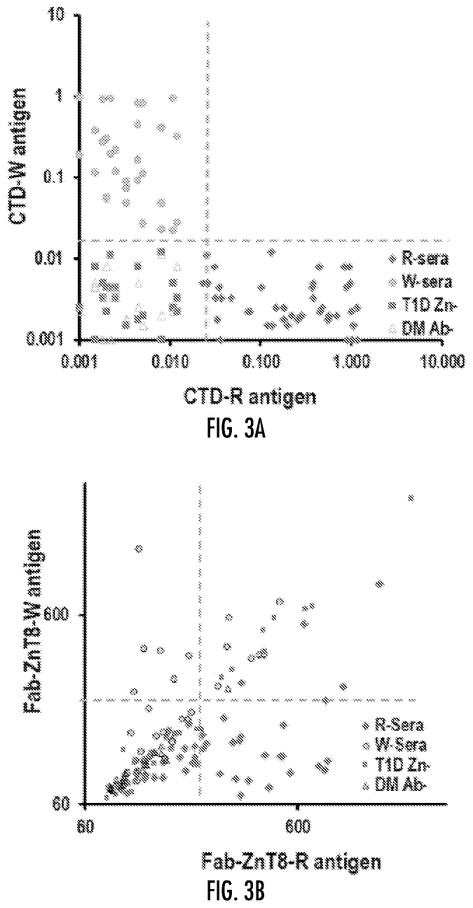

Assay validation. To eliminate potential positivity from incomplete blockade of CTDA signals, the present inventors used CTDA negative sera to validate the ECL assay. As such, a positive ECL readout can be definitely assigned to TMDAs. It is well established that serum CTDAs exclusively react either with R or W residue at position 325 (19-21). Sera from R-form homozygous patients (termed R-sera) do not cross-react with the CTD-W variant, while sera from W-form homozygous patients (termed W-sera) do not cross-react with the CTD-R variant. Sera from R/W heterozygous patients (termed R/W sera) can react with both CTD-R and CTD-W antigens. Since the TMDA epitope on the surface of TMD is independent of polymorphic variations in CTD, a bode fide TMDA in either R or W-sera is expected to cross-react with both flZnT8-R and flZnT8-W variant (fl=full length). With this expectation, the present inventors tested 320 serum samples, including 96 from new onset patients with T1D, 22 from new onset diabetic patients with all AA negative, and 182 from age and gender matched healthy controls from the general population. All of the 96 serum samples from T1D patients were pre-screened using CTD-R and CTD-W radioimmunoassay (RIA), showing that each serum was exclusively CTD-R or CTD-W positive, or negative for both CTD variants (FIG. 3A). Next, the present inventors generated two different Fab-flZnT8 complexes with an R or W variant, and tested each serum sample for cross-reaction with both variants. R/W cross-reactivity indicated TMDA positivity, yielding a subclass of diagonal datapoints in the R-to-W plot (FIG. 3B). With the cut-off set to the 98th percentile using 182 control subjects (magenta dashed lines), TMDAs were identified in 21% of T1D patients (20/96: 5/37 of R-sera, 7/24 of W-sera, and 8/35 of CTD-ZnT8A negative sera) and 1/22 of diabetic patients with all AA negative (FIG. 3B). The 21% prevalence of TMD-ZnT8As estimated by the ECL assay was consistent with the ˜17% prevalence estimated by the pGOLD assay that used a completely different antigen formula (proteoliposomes/ZnT8-Fab complex), serum set (IASP-UF/UC), assay platform (solid/solution) and detection method (fluorescence/ECL). Of note, the frequency of TMD-ZnT8As in patients with negative CTDA is approximately equal to that of patients with positive CTDAs, both having over 20% positivity. Thus, TMDA appear to be a novel autoimmunity marker independent of the well-established CTDA.

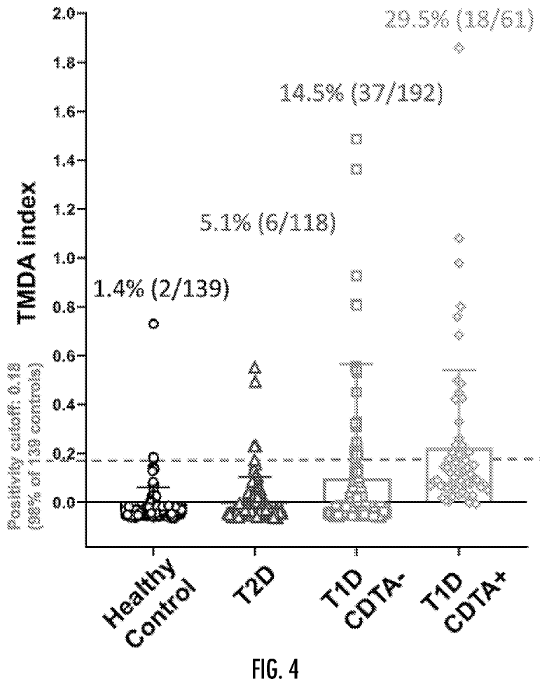

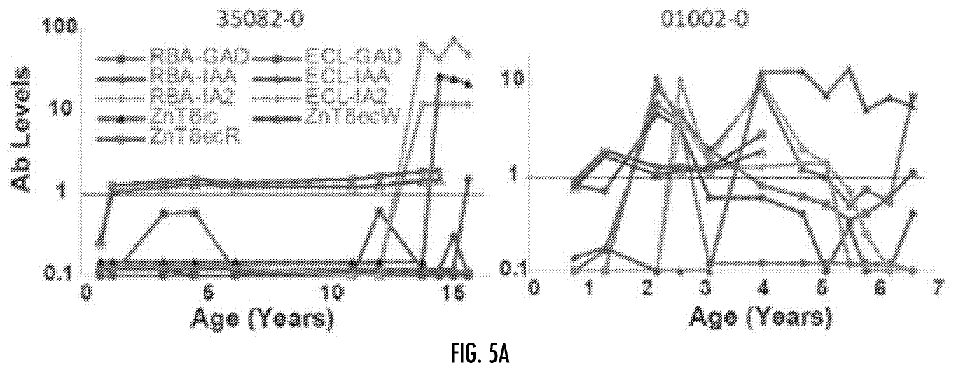

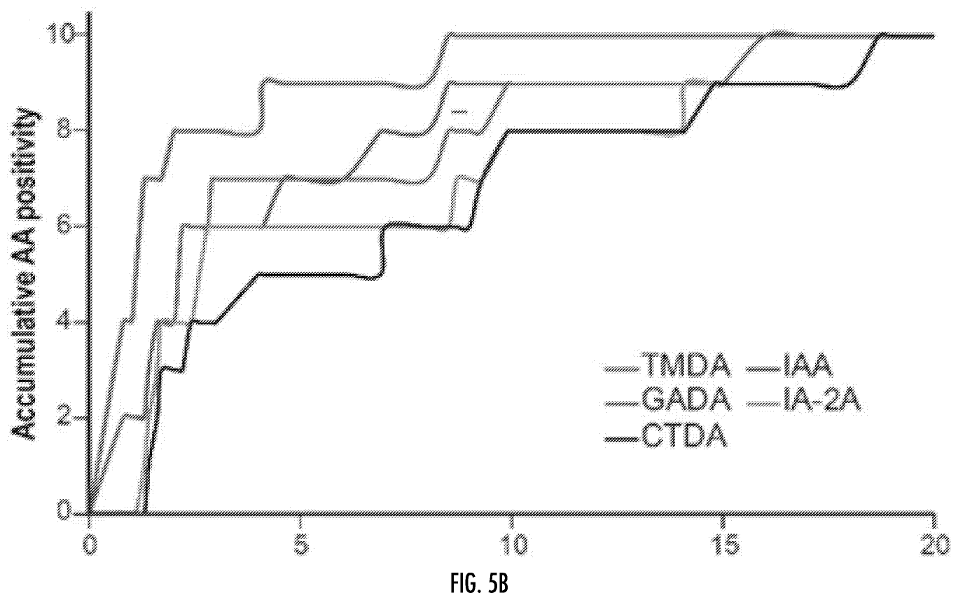

TMDA prevalence. The present inventors extended the analysis to patient cohorts with T2D and T1D, respectively. Sera from patients with T1D were pre-screened for CTDA positivity using RIA with CTD-R/W dimer as a testing antigen. Based on the screen result, the T1D cohort was further divided into CTDA negative (both CTD-R and CTD-W AA negative) and positive subgroups (either CTD-R or CYD-W positive). A TMDA positivity cut-off was set to the 98th percentile of 139 healthy controls (2/139) included subjects from ASK study (general population aged 2-17 y in Denver with none of islet autoantibodies positive), organ donor (non-DM and all AA negative), and DAISY controls (T1D first relatives or susceptible subjects with high risk HLA, but non-DM and AA developed during 10-20 y follow-up). The rates of TMDA prevalence in T2D, T1D with CTDA negative and T1D with CTDA positive cohorts were 5.1% (6/118), 14.7% (37/192) and 29.5 (18/61), respectively (FIG. 4).

First appearance of TMDA. In this Example, the present inventors analyze cases that are longitudinally followed from birth to clinical T1D or to multiple islet AA positive from, for example, the Diabetes Autoimmunity Study in the Young (DAISY). In this cohort, many children seroconvert from AA negative to sequentially positive for multiple islet AAs. It is expected that TMDA appears earlier than CTDA, GAD65 and IA2. A rank order of AA first appearance during the progression of humoral response to overt T1D is identified.

1. Ziegler, A. G., Rewers, M., Simell, O., Simell, T., Lempainen, J., Steck, A., Winkler, C., Ilonen, J., Veijola, R., Knip, M., Bonifacio, E., and Eisenbarth, G. S. (2013) Seroconversion to multiple islet autoantibodies and risk of progression to diabetes in children. JAMA 309, 2473-2479.

2. Steck, A. K., Vehik, K., Bonifacio, E., Lernmark, A., Ziegler, A. G., Hagopian, W. A., She, J., Simell, O., Akolkar, B., Krischer, J., Schatz, D., Rewers, M. J., and Group, T. S. (2015) Predictors of Progression From the Appearance of Islet Autoantibodies to Early Childhood Diabetes: The Environmental Determinants of Diabetes in the Young (TEDDY). Diabetes Care 38, 808-813.

3. Klingensmith, G. J., Pyle, L., Arslanian, S., Copeland, K. C., Cuttler, L., Kaufman, F., Laffel, L., Marcovina, S., Tollefsen, S. E., Weinstock, R. S., Linder, B., and Group, T. S. (2010) The presence of GAD and IA-2 antibodies in youth with a type 2 diabetes phenotype: results from the TODAY study. Diabetes Care 33, 1970-1975.

4. Long, A. E., Gillespie, K. M., Rokni, S., Bingley, P. J., and Williams, A. J. (2012) Rising incidence of type 1 diabetes is associated with altered immunophenotype at diagnosis. Diabetes 61, 683-686.

5. Wenzlau, J. M., and Hutton, J. C. (2013) Novel diabetes autoantibodies and prediction of type 1 diabetes. Curr Diab Rep 13, 608-615.

6. Merriman, C., Huang, Q., Rutter, G. A., and Fu, D. (2016) Lipid-tuned Zinc Transport Activity of Human ZnT8 Protein Correlates with Risk for Type-2 Diabetes. J Biol Chem 291, 26950-26957.

7. Wan, H., Merriman, C., Atkinson, M. A., Wasserfall, C. H., McGrail, K. M., Liang, Y., Fu, D., and Dai, H. (2017) Proteoliposome-based full-length ZnT8 self-antigen for type 1 diabetes diagnosis on a plasmonic platform. Proc Natl Acad Sci USA 114, 10196-10201.

8. Segerstolpe, A., Palasantza, A., Eliasson, P., Andersson, E. M., Andreasson, A. C., Sun, X., Picelli, S., Sabirsh, A., Clausen, M., Bjursell, M. K., Smith, D. M., Kasper, M., Ammala, C., and Sandberg, R. (2016) Single-Cell Transcriptome Profiling of Human Pancreatic Islets in Health and Type 2 Diabetes. Cell Metab 24, 593-607.

9. Chimienti, F., Devergnas, S., Favier, A., and Seve, M. (2004) Identification and cloning of a beta-cell-specific zinc transporter, ZnT-8, localized into insulin secretory granules. Diabetes 53, 2330-2337.

10. Huang, Q., Merriman, C., Zhang, H., and Fu, D. (2017) Coupling of Insulin Secretion and Display of a Granule-resident Zinc Transporter ZnT8 on the Surface of Pancreatic Beta Cells. J Biol Chem 292, 4034-4043.

11. Pietropaolo, M., Surhigh, J. M., Nelson, P. W., and Eisenbarth, G. S. (2008) Primer: immunity and autoimmunity. Diabetes 57, 2872-2882.

12. Yu, L., Rewers, M., Gianani, R., Kawasaki, E., Zhang, Y., Verge, C., Chase, P., Klingensmith, G., Erlich, H., Norris, J., and Eisenbarth, G. S. (1996) Antiislet autoantibodies usually develop sequentially rather than simultaneously. J Clin Endocrinol Metab 81, 4264-4267.

13. Ilonen, J., Hammais, A., Laine, A. P., Lempainen, J., Vaarala, 0., Veijola, R., Simell, O., and Knip, M. (2013) Patterns of beta-cell autoantibody appearance and genetic associations during the first years of life. Diabetes 62, 3636-3640.

14. Merriman, C., Huang, Q., Gu, W., Yu, L., and Fu, D. (2018) A subclass of serum anti-ZnT8 antibodies directed to the surface of live pancreatic beta-cells. JBiol Chem 293, 579-587.

15. Aryan, P., Pietropaolo, M., Ostrov, D., and Rhodes, C. J. (2012) Islet autoantigens: structure, function, localization, and regulation. Cold Spring Harb Perspect Med 2.

16. Kodama, K., Zhao, Z., Toda, K., Yip, L., Fuhlbrigge, R., Miao, D., Fathman, C. G., Yamada, S., Butte, A. J., and Yu, L. (2016) Expression-Based Genome-Wide Association Study Links Vitamin D-Binding Protein With Autoantigenicity in Type 1 Diabetes. Diabetes 65, 1341-1349.

17. Lu, M., and Fu, D. (2007) Structure of the zinc transporter YiiP. Science. 317, 1746-1748.

18. Merriman, C., Li, H., Li, H., and Fu, D. (2018) Highly specific monoclonal antibodies for allosteric inhibition and immunodetection of the human pancreatic zinc transporter ZnT8. J Biol Chem 293, 16206-16216.

19. Wenzlau, J. M., Liu, Y., Yu, L., Moua, 0., Fowler, K. T., Rangasamy, S., Walters, J., Eisenbarth, G. S., Davidson, H. W., and Hutton, J. C. (2008) A common nonsynonymous single nucleotide polymorphism in the SLC30A8 gene determines ZnT8 autoantibody specificity in type 1 diabetes. Diabetes 57, 2693-2697.

20. Wenzlau, J. M., Frisch, L. M., Hutton, J. C., Fain, P. R., and Davidson, H. W. (2015) Changes in Zinc Transporter 8 Autoantibodies Following Type 1 Diabetes Onset: The Type 1 Diabetes Genetics Consortium Autoantibody Workshop. Diabetes Care 38 Suppl 2, S14-20.

21. Skarstrand, H., Krupinska, E., Haataja, T. J., Vaziri-Sani, F., Lagerstedt, J. O., and Lernmark, A. (2015) Zinc transporter 8 (ZnT8) autoantibody epitope specificity and affinity examined with recombinant ZnT8 variant proteins in specific ZnT8R and ZnT8W autoantibody-positive type 1 diabetes patients. Clin Exp Immunol 179, 220-229.

### Example 2: Longitudinal Studies of Autoantibodies to the Transmembrane Domain of Human ZnT8 in Children Progressing to Type-1 Diabetes

Type 1 diabetes (T1D) is an autoimmune disease characterized by the pancreatic infiltration of immune cells, resulting in autoimmune destruction of the insulin-producing β cells. Innate immune cells infiltrate the pancreas first, releasing proinflammatory cytokines and chemokines that activate hyper-expression of human leucocyte antigen (HLA) class I molecules on the β cells and attract autoreactive B and T lymphocytes into the islets (1). B cells make autoantibodies (AAs) against islet autoantigens. Since seroconversion of AAs is triggered months and most often years prior to the onset of T1D (2), AAs are biomarkers for autoimmune responses. However, none of the islet AAs identified so far react with epitopes on the cell surface to exert direct cytotoxic effects. In previous studies with the ECL assay, the present inventors identified AAs directed to the transmembrane domain (TMD) of ZnT8 expressed on the surface of live β cells. Here, the present inventors report additional results in determining how early TMDAs appear in pre-T1D children. A prevalent view of T1D autoimmunity development is that earliest autoantibodies (AAs) in humans are predominantly directed to insulin (3,4), and then spread over time to GAD65, IA-2 and ZnT8 (CTD) as secondary antigens following β cell injury or activation (5). However, this view is challenged by the present inventors' longitudinal study of AAs during progression to T1D in 10 subjects of the diabetes autoimmunity study in the young (DAISY). The present inventors found that TMDAs were the first AA expressed in all 10 children during a median follow-up of 7.0 years. Notably, TMDAs in all children occurred earlier or at the same time of first insulin autoantibody (IAA) appearance. There is a median delay of 4.3 years between the first appearances of TMDAs and CTDAs, suggesting that TMDAs, like IAAs, may be involved in initiation of islet autoimmunity while CTDAs may reflect epitope spreading. This preliminary observation is consistent with earlier findings that TMDA and CTDA are two distinct subclasses of ZnT8As directed to TMD on the cell surface and CTD in the cytoplasm, respectively (6).

### References

1. Atkinson, M. A., Bluestone, J. A., Eisenbarth, G. S., Hebrok, M., Herold, K. C., Accili, D., Pietropaolo, M., Aryan, P. R., Von Herrath, M., Markel, D. S., and Rhodes, C. J. (2011) How does type 1 diabetes develop?: the notion of homicide or beta-cell suicide revisited. Diabetes 60, 1370-1379

2. Ferrannini, E., Mari, A., Nofrate, V., Sosenko, J. M., Skyler, J. S., and Group, D. P. T. S. (2010) Progression to diabetes in relatives of type 1 diabetic patients: mechanisms and mode of onset. Diabetes 59, 679-685

3. Yu, L., Dong, F., Miao, D., Fouts, A. R., Wenzlau, J. M., and Steck, A. K. (2013) Proinsulin/Insulin autoantibodies measured with electrochemiluminescent assay are the earliest indicator of prediabetic islet autoimmunity. Diabetes Care 36, 2266-2270

4. Yu, L., Robles, D. T., Abiru, N., Kaur, P., Rewers, M., Kelemen, K., and Eisenbarth, G. S. (2000) Early expression of antiinsulin autoantibodies of humans and the NOD mouse: evidence for early determination of subsequent diabetes. Proc Natl Acad Sci USA 97, 1701-1706

5. Pietropaolo, M., Surhigh, J. M., Nelson, P. W., and Eisenbarth, G. S. (2008) Primer: immunity and autoimmunity. Diabetes 57, 2872-2882

6. Merriman, C., Huang, Q., Gu, W., Yu, L., and Fu, D. (2018) A subclass of serum anti-ZnT8 antibodies directed to the surface of live pancreatic beta-cells. J Biol Chem 293, 579-587

1. A method comprising the steps of:
(a) contacting in a first mixture a biological sample obtained from a patient with a zinc transporter 8 (ZnT8)-antibody complex, wherein the ZnT8-antibody complex comprises ZnT8 and at least one detectably labeled anti-ZnT8 antibody or antigen-binding fragment thereof that specifically binds to the cytoplasmic domain of ZnT8;
(b) contacting in a second mixture the first mixture of step (a) with an immunoglobulin G (IgG) labeled with a tag molecule;
(c) contacting the second mixture of step (b) with a solid substrate coated with a capture molecule that specifically binds the tag molecule; and
(d) detecting a signal emitted from the detectably labeled anti-ZnT8 antibody or antigen-binding fragment thereof.

2. The method of claim 1, wherein the biological sample is blood, plasma or serum.

3. The method of claim 1, wherein the at least one detectably labeled anti-ZnT8 antibody or antigen-binding fragment thereof comprises a Fab.

4. The method of claim 3, wherein the Fab comprises SEQ ID NO:32 and SEQ ID NO:37.

5. The method of claim 3, wherein the Fab comprises:
(a) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions; and
(b) light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions.

6. The method of claim 3, wherein the Fab comprises SEQ ID NO:52 and SEQ ID NO:57.

7. The method of claim 3, wherein the Fab comprises:
(a) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions; and
(b) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

8. The method of claim 1, wherein the at least one detectably labeled anti-ZnT8 antibody or antigen-binding fragment thereof comprises (a) a first Fab comprising SEQ ID NO:32 and SEQ ID NO:37; and a second Fab comprising SEQ ID NO:52 and SEQ ID NO:57.

9. The method of claim 1, wherein the at least one detectably labeled anti-ZnT8 antibody or antigen-binding fragment thereof comprises:
(a) a first Fab comprising:
(i) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions, and
(ii) light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions; and
(b) a second Fab comprising:
(i) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions, and
(ii) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

10. The method of claim 1, wherein ZnT8 is full length ZnT8.

11. The method of claim 1, wherein ZnT8 lacks an N-terminal domain.

12. The method of claim 1, wherein ZnT8 comprises amino acids 66-369 of SEQ ID NO:64.

13. The method of claim 1, wherein the detectable label is an electrochemiluminescent label.

14. The method of claim 13, wherein the electrochemiluminescent label is a sulfo-tag.

15. The method of claim 1, wherein the cytoplasmic domain of ZnT8 comprises amino acids 276-369 of SEQ ID NO:64.

16. The method of claim 1, wherein the tag molecule is biotin.

17. The method of claim 1, wherein the capture molecule is streptavidin.

18. A method comprising the steps of:
(a) contacting in a mixture a biological sample obtained from a patient with (i) a ZnT8-antibody complex, wherein the ZnT8-antibody complex comprises ZnT8 and at least one detectably labeled anti-ZnT8 antibody or antigen-binding fragment thereof that specifically binds to the cytoplasmic domain of ZnT8, and (ii) an immunoglobulin G (IgG) labeled with a tag molecule;
(b) contacting the mixture of step (a) with a solid substrate coated with a capture molecule that specifically binds the tag molecule; and
(c) detecting a signal emitted from the detectably labeled anti-ZnT8 antibody or antigen-binding fragment thereof.

19. A ZnT8-antibody complex comprising:
(a) ZnT8; and
(b) at least one anti-ZnT8 antibody or antigen-binding fragment thereof that specifically binds to the cytoplasmic domain of ZnT8.

20. The ZnT8-antibody complex of claim 19, wherein ZnT8 is full length ZnT8.

21. The ZnT8-antibody complex of claim 19, wherein ZnT8 lacks an N-terminal domain.

22. The ZnT8-antibody complex of claim 19, wherein ZnT8 comprises amino acids 66-369 of SEQ ID NO:64.

23. The ZnT8-antibody complex of claim 19, wherein the at least one anti-ZnT8 antibody or antigen-binding fragment thereof comprises a Fab.

24. The ZnT8-antibody complex of claim 23, wherein the Fab comprises SEQ ID NO:32 and SEQ ID NO:37.

25. The ZnT8-antibody complex of claim 23, wherein the Fab comprises:
(a) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions; and
(b) light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions.

26. The ZnT8-antibody complex of claim 23, wherein the Fab comprises SEQ ID NO:52 and SEQ ID NO:57.

27. The ZnT8-antibody complex of claim 23, wherein the Fab comprises:
(a) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions; and
(b) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

28. The ZnT8-antibody complex of claim 19, wherein the at least one detectably labeled anti-ZnT8 antibody or antigen-binding fragment thereof comprises (a) a first Fab comprising SEQ ID NO:32 and SEQ ID NO:37; and a second Fab comprising SEQ ID NO:52 and SEQ ID NO:57.

29. The ZnT8-antibody complex of claim 19, wherein the at least one detectably labeled anti-ZnT8 antibody or antigen-binding fragment thereof comprises:
(a) a first Fab comprising:
(i) heavy chain complementary determining regions (CDRs) 1, 2, and 3, wherein the heavy chain CDR1 comprises SEQ ID NO:33, or the amino acid sequence of SEQ ID NO:33 with a substitution at two or fewer amino acid positions, the heavy chain CDR2 comprises SEQ ID NO:34, or the amino acid sequence of SEQ ID NO:34 with a substitution at two or fewer amino acid positions, and the heavy chain CDR3 comprises SEQ ID NO:35, or the amino acid sequence of SEQ ID NO:35 with a substitution at two or fewer amino acid positions, and
(ii) light chain CDRs 1, 2, and 3, wherein the light chain CDR1 comprises SEQ ID NO:38, or the amino acid sequence of SEQ ID NO:38 with a substitution at two or fewer amino acid positions, the light chain CDR2 comprises SEQ ID NO:39, or the amino acid sequence of SEQ ID NO:39 with a substitution at two or fewer amino acid positions, and the light chain CDR3 comprises SEQ ID NO:40, or the amino acid sequence of SEQ ID NO:40 with a substitution at two or fewer amino acid positions; and
(b) a second Fab comprising:
(i) heavy chain complementary determining regions (CDRs) 1, 2, and 3, wherein the heavy chain CDR1 comprises SEQ ID NO:53, or the amino acid sequence of SEQ ID NO:53 with a substitution at two or fewer amino acid positions, the heavy chain CDR2 comprises SEQ ID NO:54, or the amino acid sequence of SEQ ID NO:54 with a substitution at two or fewer amino acid positions, and the heavy chain CDR3 comprises SEQ ID NO:55, or the amino acid sequence of SEQ ID NO:55 with a substitution at two or fewer amino acid positions, and
(ii) light chain CDRs 1, 2, and 3, wherein the light chain CDR1 comprises SEQ ID NO:58, or the amino acid sequence of SEQ ID NO:58 with a substitution at two or fewer amino acid positions, the light chain CDR2 comprises SEQ ID NO:59, or the amino acid sequence of SEQ ID NO:59 with a substitution at two or fewer amino acid positions, and the light chain CDR3 comprises SEQ ID NO:60, or the amino acid sequence of SEQ ID NO:60 with a substitution at two or fewer amino acid positions.

30. The ZnT8-antibody complex of claim 19, wherein the at least one anti-ZnT7 antibody or antigen-binding fragment thereof is detectably labeled.

31. The ZnT8-antibody complex of claim 30, wherein the label is an ECL label.

32. The ZnT8-antibody complex of claim 31, wherein the ECL label is a sulfo-tag.


SEQUENCE LISTING (64 sequences)

SEQ ID NO: 1 - DNA, 1404 bp, Artificial Sequence
  mAB12 heavy chain
atggacatgagggtgcccgctcagctcctggggctcctgctgctgtggctgagaggtgcgcgctgtcagatccagttggtgcagtctggacctgagctgaagaagcctggagagacagtcaagatctcctgcaaggcttctgggtataccttcacaaactatccaatgcactgggtgaagcagactccaggaaagggtttaaagtggatgggctggataaacacctactctggagtgccaacatatggagatgacttcaagggacggtttgccttctctttggaaacctctgccagtactgcatatttgcagatcaacaacctcaaaaatgaagacatggctacatatttctgtgcaagatcgaacccctatgattacttgtatgctatggactcctggggtcaaggaacctcagtcaccgtctctagtgccaaaacgacacccccatctgtctacccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 2 - protein, 467 aa, Artificial Sequence
  mAB12 heavy chain
MDMRVPAQLLGLLLLWLRGARCQIQLVQSGPELKKPGETVKISCKASGYTFTNYPMHWVKQTPGKGLKWMGWINTYSGVPTYGDDFKGRFAFSLETSASTAYLQINNLKNEDMATYFCARSNPYDYLYAMDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 3 - protein, 7 aa, Artificial Sequence
  mAb12 heavy chain CDR1
GYTFTNY

SEQ ID NO: 4 - protein, 5 aa, Artificial Sequence
  mAb12 heavy chain CDR2
TYSGV

SEQ ID NO: 5 - protein, 12 aa, Artificial Sequence
  mAB heavy chain CDR3
SNPYDYLYAMDS

SEQ ID NO: 6 - DNA, 726 bp, Artificial Sequence
  mAb12 light chain
atggacatgagggtgcccgctcagctcctggggctcctgctgctgtggctgagaggtgcgcgctgtgatgttgtgatgacccaaactccactctccctgcctgtcagtcttggagatcaagcctccatctcctgcagatctagtcagagccttgtacacatcaatggaaacacctatatacattggtacctgcagaagccaggccagtctccaaagctcctgatctacaaagtttccaaccgattttctggggtcccagacaggttcagtggcagtggatcagggacagattttacactcaagatcagaagagtggaggctgaggatctgggagtttatttctgctctcaaaatacacatgttccattcacattcggctcggggacaaagttggaaataaaacgtgcagatgctgcgccaactgtatccatcttcccaccatctagcgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 7 - protein, 241 aa, Artificial Sequence
  mAb12 light chain
MDMRVPAQLLGLLLLWLRGARCDVVMTQTPLSLPVSLGDQASISCRSSQSLVHINGNTYIHWYLQKPGQSPKLLIYKVSNRFSGVPDRFSGSGSGTDFTLKIRRVEAEDLGVYFCSQNTHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 16 aa, Artificial Sequence
  mAb12 light chain CDR1
RSSQSLVHINGNTYIH

SEQ ID NO: 9 - protein, 7 aa, Artificial Sequence
  mAb12 light chain CDR2
KVSNRFS

SEQ ID NO: 10 - protein, 9 aa, Artificial Sequence
  mAb12 light chain CDR3
SQNTHVPFT

SEQ ID NO: 11 - DNA, 1401 bp, Artificial Sequence
  mAb16 heavy chain
atggacatgagggtgcccgctcagctcctggggctcctgctgctgtggctgagaggtgcgcgctgtcagatccagttggtgcagtctggacctgagctgaagaagcctggagagacagtcacgatctcctgcaaggcttctggatataccttcacacactatccagtgcactgggtgaagcaggctccaggaaagggtttacagtggatgggctggataaacacctactctggagtgccaacatatgcagatgccttcaagaaacgttttgccttctctttggaaacctctgccagcactgcatatttgcagatcaacaacctcaaaagtgaggacatggctacatatttctgtgcaagatcgagggtctatgatgggtactattttgactactggggccaaggcaccactctcaccgtctctagtgccaaaacgacacccccatctgtctacccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 12 - protein, 466 aa, Artificial Sequence
  mAb16 heavy chain
MDMRVPAQLLGLLLLWLRGARCQIQLVQSGPELKKPGETVTISCKASGYTFTHYPVHWVKQAPGKGLQWMGWINTYSGVPTYADAFKKRFAFSLETSASTAYLQINNLKSEDMATYFCARSRVYDGYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - protein, 7 aa, Artificial Sequence
  mAb16 heavy chain CDR1
GYTFTHY

SEQ ID NO: 14 - protein, 6 aa, Artificial Sequence
  mAb16 heavy chain CDR2
NTYSGV

SEQ ID NO: 15 - protein, 11 aa, Artificial Sequence
  mAb16 heavy chain CDR3
SRVYDGYYFDY

SEQ ID NO: 16 - DNA, 726 bp, Artificial Sequence
  mAb16 light chain
atggacatgagggtgcccgctcagctcctggggctcctgctgctgtggctgagaggtgcgcgctgtgatgttgtgatgacccaaactccactctccctgcctgtcagtcttggagatcaagcctccatctcttgcagatctagtcagagccttgtacacagtaatggaaagacctatttacattggtacctgcagaagccaggccagtctccaaacctcctgatctacaaagtttccaaccgattttctggggtcccagacaggttcagtggcagtggatcagggacagatttcacactcaagatcagcagagtggaggctgaggatctgggagtttatttctgctctcaacttacacatgttccgtggacgttcggtggaggcaccaagctggaaatcaaacgtgcagatgctgcgccaactgtatccatcttcccaccatctagcgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 17 - protein, 241 aa, Artificial Sequence
  mAb16 light chain
MDMRVPAQLLGLLLLWLRGARCDVVMTQTPLSLPVSLGDQASISCRSSQSLVHSNGKTYLHWYLQKPGQSPNLLIYKVSNRFSGVPDRFSGSGSGTDFTLKISRVEAEDLGVYFCSQLTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - protein, 16 aa, Artificial Sequence
  mAb16 light chain CDR1
RSSQSLVHSNGKTYLH

SEQ ID NO: 19 - protein, 7 aa, Artificial Sequence
  mAb16 light chain CDR2
KVSNRFS

SEQ ID NO: 20 - protein, 8 aa, Artificial Sequence
  mAb16 light chain CDR3
SQLTHVPW

SEQ ID NO: 21 - DNA, 1407 bp, Artificial Sequence
  mAb17 heavy chain
atggacatgagggtgcccgctcagctcctggggctcctgctgctgtggctgagaggtgcgcgctgtcagatccagttggtgcagtctggacctgagctgaagaagcctggagagacagtcaagatctcctgcaaggcttctgggtataccttcacaaactatccaatgcactggttgaagcaggctccaggaaagggtttaaagtggatgggctggataaacacctactctggagtgccaacatatgcagatgacttcaagggacggtttgccttctctttggaaacctctgccagcactgcatatttgcagatcaacaacctcaaaaatgaggacatggctacatatttctgtacaaaatcgcgcattactacgatggggggttatgctatggactgctggggtcaaggaacctcagtcaccgtctctagtgccaaaacgacacccccatctgtctacccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 22 - protein, 468 aa, Artificial Sequence
  mAb17 heavy chain
MDMRVPAQLLGLLLLWLRGARCQIQLVQSGPELKKPGETVKISCKASGYTFTNYPMHWLKQAPGKGLKWMGWINTYSGVPTYADDFKGRFAFSLETSASTAYLQINNLKNEDMATYFCTKSRITTMGGYAMDCWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 23 - protein, 7 aa, Artificial Sequence
  mAb17 heavy chain CDR1
GYTFTNY

SEQ ID NO: 24 - protein, 6 aa, Artificial Sequence
  mAb17 heavy chain CDR2
NTYSGV

SEQ ID NO: 25 - protein, 13 aa, Artificial Sequence
  mAb17 heavy chain CDR3
SRITTMGGYAMDC

SEQ ID NO: 26 - DNA, 726 bp, Artificial Sequence
  mAb17 light chain
atggacatgagggtgcccgctcagctcctggggctcctgctgctgtggctgagaggtgcgcgctgtgaagttgtgatgacccaaactccactctccctgcctgtcagtcttggagatcaagcctccatctcttgcagatctagtcagagccttctacacagtaatggaaacacctatttacattggtacctgcagaggccaggccagtctccaaacctcctgatctccaaagtttccaaccgattttctggggtcccagacaggttcagtggcagtggatcagggacagatttcacactcaagatcagcagagtggaggctgaggatctgggagtttatttctgctctcaaaatacacatgttccgtggacgttcggtggaggcaccaagctggaaatcaaacgtgcagatgctgcgccaactgtatccatcttcccaccatctagcgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 27 - protein, 241 aa, Artificial Sequence
  mAb17 light chain
MDMRVPAQLLGLLLLWLRGARCEVVMTQTPLSLPVSLGDQASISCRSSQSLLHSNGNTYLHWYLQRPGQSPNLLISKVSNRFSGVPDRFSGSGSGTDFTLKISRVEAEDLGVYFCSQNTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 28 - protein, 16 aa, Artificial Sequence
  mAb17 light chain CDR1
RSSQSLLHSNGNTYLH

SEQ ID NO: 29 - protein, 7 aa, Artificial Sequence
  mAb17 light chain CDR2
KVSNRFS

SEQ ID NO: 30 - protein, 9 aa, Artificial Sequence
  mAb17 light chain CDR3
SQNTHVPWT

SEQ ID NO: 31 - DNA, 1407 bp, Artificial Sequence
  mAb20 heavy chain
atggacatgagggtgcccgctcagctcctggggctcctgctgctgtggctgagaggtgcgcgctgtcagatccagttggtgcagtctggacctgagctgaagaagcctggagagacagtcaagatctcctgcaaggcttctgggtatatcttcacaaactatccaatgcactgggtgaagcaggctccaggaaagggtttaaagtggatgggctggataaacacctactctggagtgccaacacatgcagatgacttcaagggacggtttgccttctctttggaaacctctgccaacagtgcatttttgcagatcaacaacctcaaaaatgaggacacggctacatatttctgtacaagatcgcgcattactccgacggggggctatgctatggactactggggtcaaggaacctcagtcaccgtctctagtgccaaaacgacacccccatctgtctacccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 32 - protein, 468 aa, Artificial Sequence
  mAb20 heavy chain
MDMRVPAQLLGLLLLWLRGARCQIQLVQSGPELKKPGETVKISCKASGYIFTNYPMHWVKQAPGKGLKWMGWINTYSGVPTHADDFKGRFAFSLETSANSAFLQINNLKNEDTATYFCTRSRITPTGGYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 33 - protein, 7 aa, Artificial Sequence
  mAb20 heavy chain CDR1
GYIFTNY

SEQ ID NO: 34 - protein, 6 aa, Artificial Sequence
  mAb20 heavy chain CDR2
NTYSGV

SEQ ID NO: 35 - protein, 13 aa, Artificial Sequence
  mAb20 heavy chain CDR3
SRITPTGGYAMDY

SEQ ID NO: 36 - DNA, 726 bp, Artificial Sequence
  mAb20 light chain
atggacatgagggtgcccgctcagctcctggggctcctgctgctgtggctgagaggtgcgcgctgtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaacctcctgatctccaaagtttccaaccgattttctggggtcccagaaaggttcagtggcagtggatcagggacagatttcacactcaagatcagcagagtggaggctgaggatctgggagtttatttctgctctcaaaatacacatgttccgtggacgttcggtggaggcaccaagctggaaatcaaacgtgcagatgctgcgccaactgtatccatcttcccaccatctagcgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 37 - protein, 241 aa, Artificial Sequence
  mAb20 light chain
MDMRVPAQLLGLLLLWLRGARCDVVMTQTPLSLPVSLGDQASISCRSSQSLVHSNGNTYLHWYLQKPGQSPNLLISKVSNRFSGVPERFSGSGSGTDFTLKISRVEAEDLGVYFCSQNTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 16 aa, Artificial Sequence
  mAb20 light chain CDR1
RSSQSLVHSNGNTYLH

SEQ ID NO: 39 - protein, 7 aa, Artificial Sequence
  mAb20 light chain CDR2
KVSNRFS

SEQ ID NO: 40 - protein, 9 aa, Artificial Sequence
  mAb40 light chain CDR3
SQNTHVPWT

SEQ ID NO: 41 - DNA, 1407 bp, Artificial Sequence
  mAb28 heavy chain
atggacatgagggtgcccgctcagctcctggggctcctgctgctgtggctgagaggtgcgcgctgtcagatccagttggtgcagtctggacctgagctgaagaagcctggagagacagtcaagatctcctgcaaggcttctgggtataccttcacaaactatccaatgcactgggtaaagcaggctccaggaaaggatttaaagtggatgggctggataaacacctactctggaatgtcaacatatgcagatgacttcaagggacggtttgccttctctttggaaacctctgccagcactgcgtatttgcagatcaacaacctcaaaaatgaggacatggctacatatttctgtgcaagatcgcgcattacaacgatggggggctatgctatggactactggggtcaaggagcctcagtcaccgtctctagtgccaaaacgacacccccatctgtctacccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 42 - protein, 468 aa, Artificial Sequence
  mAb28 heavy chain
MDMRVPAQLLGLLLLWLRGARCQIQLVQSGPELKKPGETVKISCKASGYTFTNYPMHWVKQAPGKDLKWMGWINTYSGMSTYADDFKGRFAFSLETSASTAYLQINNLKNEDMATYFCARSRITTMGGYAMDYWGQGASVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 43 - protein, 7 aa, Artificial Sequence
  mAb28 heavy chain CDR1
GYTFTNY

SEQ ID NO: 44 - protein, 6 aa, Artificial Sequence
  mAb28 heavy chain CDR2
NTYSGM

SEQ ID NO: 45 - protein, 13 aa, Artificial Sequence
  mAb28 heavy chain CDR3
SRITTMGGYAMDY

SEQ ID NO: 46 - DNA, 726 bp, Artificial Sequence
  mAb28 light chain
atggacatgagggtgcccgctcagctcctggggctcctgctgctgtggctgagaggtgcgcgctgtgaagttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctccaaagtttccaaccgattttctggggtcccagacaggttcagtggcagtggatcagggacagatttcacactcaagatcatcagagtggaggctgaggatctgggagtttatttctgctctcaaaatacacatgttccgtggacgttcggtggaggcaccaagctggaaatcaaacgtgcagatgctgcgccaactgtatccatcttcccaccatctagcgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 47 - protein, 241 aa, Artificial Sequence
  mAb28 light chain
MDMRVPAQLLGLLLLWLRGARCEVVMTQTPLSLPVSLGDQASISCRSSQSLVHSNGNTYLHWYLQKPGQSPKLLISKVSNRFSGVPDRFSGSGSGTDFTLKIIRVEAEDLGVYFCSQNTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 48 - protein, 16 aa, Artificial Sequence
  mAb28 light chain CDR1
RSSQSLVHSNGNTYLH

SEQ ID NO: 49 - protein, 7 aa, Artificial Sequence
  mAb28 light chain CDR2
KVSNRFS

SEQ ID NO: 50 - protein, 9 aa, Artificial Sequence
  mAb28 light chain CDR3
SQNTHVPWT

SEQ ID NO: 51 - DNA, 1389 bp, Artificial Sequence
  mAb39 heavy chain
atgggatggagctgtatcatgttctttttggtagcaacagctacagatgtccactcccaggtccaactgcagcagcctggggctgaactggtgaagcctggggcttcagtgaagctgtcctgcaaggcttctggctactccttcaccacctactggatgcactgggtgaagcagaggcctggacaaggcctagagtgggttggagatattaatcctaggaacggtcgtactaactacaatgagaagtccaagagcaaggccacactgactgtagacatatcatccagcacagtatacatgcaagtcagcagcctgacatctgaggactctgcggtctattactgtgcaatatggtcgggtgctatggactactggggtccaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtgatgtgcacaccttcccagctctcctgcagtctggcctctacaccctcagcagctcagtgactgtaaccacctggcccagccagaccatcacctgcaatgtggcccacccggcaagcagcaccaaagtggacaagaaaattgagcccagagggtccccaacacataaaccctgtcctccatgcccagctcctaacctcttgggtggaccatccgtcttcatcttccctccaaagatcaaggatgtactcatgatctccctgagccccatggtcacgtgtgtggtggtggatgtgagcgaggatgacccagatgtccatgtcagctggttcgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagccctcccagcgcccatcgagagaaccatctcaaaacccaaagggccagtaagagctccacaggtatatgtcttgcctccaccagaagaagagatgactaagaaacaggtcactctgacctgcatgatcacagacttcatgcctgaagacatttacgtggagtggaccaacaacgggcaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggtctccgggtaaatga

SEQ ID NO: 52 - protein, 462 aa, Artificial Sequence
  mAb39 heavy chain
MGWSCIMFFLVATATDVHSQVQLQQPGAELVKPGASVKLSCKASGYSFTTYWMHWVKQRPGQGLEWVGDINPRNGRTNYNEKSKSKATLTVDISSSTVYMQVSSLTSEDSAVYYCAIWSGAMDYWGPGTSVTVSSAKTTAPSVYPLAPVCGDTTGSSVTLGCLVKGYFPEPVTLTWNSGSLSSDVHTFPALLQSGLYTLSSSVTVTTWPSQTITCNVAHPASSTKVDKKIEPRGSPTHKPCPPCPAPNLLGGPSVFIFPPKIKDVLMISLSPMVTCVVVDVSEDDPDVHVSWFVNNVEVHTAQTQTHREDYNSTIRVVSALPIQHQDWMSGKEFKCKVNNKALPAPIERTISKPKGPVRAPQVYVLPPPEEEMTKKQVTLTCMITDFMPEDIYVEWTNNGQTELNYKNTEPVLDSDGSYFMYSKLRVEKKNWVERNSYSCSVVHEGLHNHHTTKSFSRSPGK

SEQ ID NO: 53 - protein, 7 aa, Artificial Sequence
  mAb39 heavy chain CDR1
GYSFTTY

SEQ ID NO: 54 - protein, 6 aa, Artificial Sequence
  mAb39 heavy chain CDR2
NPRNGR

SEQ ID NO: 55 - protein, 7 aa, Artificial Sequence
  mAb39 heavy chain CDR3
WSGAMDY

SEQ ID NO: 56 - DNA, 717 bp, Artificial Sequence
  mAb39 light chain
atgaagttgcctgttaggctgttggtgctgatgttctggattcctgcttccagcagtgatgttgtgatgacccaaactccactctccctgcctgtcagtcttggagatcaaccctccatctcttgcaaatctagtcagagccttgtacacaataatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttcctccgacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 57 - protein, 238 aa, Artificial Sequence
  mAb39 light chain
MKLPVRLLVLMFWIPASSSDVVMTQTPLSLPVSLGDQPSISCKSSQSLVHNNGNTYLHWYLQKPGQSPKLLIYKVSNRFSGVPDRFSGSGSGTDFTLKISRVEAEDLGVYFCSQTTHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 58 - protein, 16 aa, Artificial Sequence
  mAb39 light chain CDR1
KSSQSLVHNNGNTYLH

SEQ ID NO: 59 - protein, 7 aa, Artificial Sequence
  mAb39 light chain CDR2
KVSNRFS

SEQ ID NO: 60 - protein, 9 aa, Artificial Sequence
  mAb39 light chain CDR3
SQTTHVPPT

SEQ ID NO: 61 - DNA, 1389 bp, Artificial Sequence
  mAb39 heavy chain v2 (t1375a-->S459T)
atgggatggagctgtatcatgttctttttggtagcaacagctacagatgtccactcccaggtccaactgcagcagcctggggctgaactggtgaagcctggggcttcagtgaagctgtcctgcaaggcttctggctactccttcaccacctactggatgcactgggtgaagcagaggcctggacaaggcctagagtgggttggagatattaatcctaggaacggtcgtactaactacaatgagaagtccaagagcaaggccacactgactgtagacatatcatccagcacagtatacatgcaagtcagcagcctgacatctgaggactctgcggtctattactgtgcaatatggtcgggtgctatggactactggggtccaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtgatgtgcacaccttcccagctctcctgcagtctggcctctacaccctcagcagctcagtgactgtaaccacctggcccagccagaccatcacctgcaatgtggcccacccggcaagcagcaccaaagtggacaagaaaattgagcccagagggtccccaacacataaaccctgtcctccatgcccagctcctaacctcttgggtggaccatccgtcttcatcttccctccaaagatcaaggatgtactcatgatctccctgagccccatggtcacgtgtgtggtggtggatgtgagcgaggatgacccagatgtccatgtcagctggttcgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagccctcccagcgcccatcgagagaaccatctcaaaacccaaagggccagtaagagctccacaggtatatgtcttgcctccaccagaagaagagatgactaagaaacaggtcactctgacctgcatgatcacagacttcatgcctgaagacatttacgtggagtggaccaacaacgggcaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 62 - protein, 462 aa, Artificial Sequence
  mAb39 heavy chain (S459T)
MGWSCIMFFLVATATDVHSQVQLQQPGAELVKPGASVKLSCKASGYSFTTYWMHWVKQRPGQGLEWVGDINPRNGRTNYNEKSKSKATLTVDISSSTVYMQVSSLTSEDSAVYYCAIWSGAMDYWGPGTSVTVSSAKTTAPSVYPLAPVCGDTTGSSVTLGCLVKGYFPEPVTLTWNSGSLSSDVHTFPALLQSGLYTLSSSVTVTTWPSQTITCNVAHPASSTKVDKKIEPRGSPTHKPCPPCPAPNLLGGPSVFIFPPKIKDVLMISLSPMVTCVVVDVSEDDPDVHVSWFVNNVEVHTAQTQTHREDYNSTIRVVSALPIQHQDWMSGKEFKCKVNNKALPAPIERTISKPKGPVRAPQVYVLPPPEEEMTKKQVTLTCMITDFMPEDIYVEWTNNGQTELNYKNTEPVLDSDGSYFMYSKLRVEKKNWVERNSYSCSVVHEGLHNHHTTKSFSRTPGK

SEQ ID NO: 63 - DNA, 1107 bp, Homo sapiens
  misc_feature (1)..(1107): znT8 coding sequence
atggagtttcttgaaagaacgtatcttgtgaatgataaagctgccaagatgtatgctttcacactagaaagtgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcaccgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgac

SEQ ID NO: 64 - protein, 369 aa, Homo sapiens
  MISC_FEATURE (1)..(369): znT8 full length amino acid sequence
  MISC_FEATURE (66)..(369): znT8 amino acid sequence without the N-terminal domain
  MISC_FEATURE (276)..(369): znT8 cytoplasmic domain
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD